(19) United States
(12) Patent Application Publication
(10) Pub. No.: US 2025/0166839 A1
(43) Pub. Date: May 22, 2025

(54) SYSTEMS AND METHODS FOR THERANOSTICS MANAGEMENT

(71) Applicant: GE Precision Healthcare LLC, Waukesha, WI (US)

(72) Inventors: Jeffrey R. Terry, Dallas, TX (US); Kathleen P. Martin, Pinckney, MI (US); Tamas Fixler, Thornhill (CA); Thomas Barrett, Cleveland, OH (US); Alexander Reeves, Houston, TX (US)

(21) Appl. No.: 18/957,527

(22) Filed: Nov. 22, 2024

Related U.S. Application Data
(60) Provisional application No. 63/602,112, filed on Nov. 22, 2023.

(51) Int. Cl. G16H 50/30; G16H 10/60; G16H 20/40; G16H 40/20

(52) U.S. Cl. CPC G16H 50/30; G16H 10/60; G16H 20/40; G16H 40/20

(57) ABSTRACT

Various methods and systems are provided for a theranostics engine for management of theranostics protocols. In one example, a computing device comprising a display screen, the computing device being configured to display on the display screen a menu listing one or more electronic medical records (EMRs) of one or more patients, and additionally being configured to display on the display screen a theranostics graphical user interface (GUI) accessible from the menu, wherein the theranostics GUI displays, for each patient, a pathway tracker indicating status of a corresponding theranostics protocol, wherein the pathway tracker comprises, for each cycle of the corresponding theranostics protocol, a limited snapshot of statuses of steps thereof, wherein each limited snapshot is selectable to launch a pop-up GUI with additional information relating to each of the steps thereof, and wherein the theranostics GUI is displayed while the one or more EMRs are in an un-launched state.

### CROSS REFERENCE TO RELATED APPLICATIONS

The present application claims priority to U.S. Provisional Application No. 63/602,112 entitled “SYSTEMS AND METHODS FOR THERANOSTICS MANAGEMENT”, and filed on Nov. 22, 2023. The entire contents of the above-listed application are hereby incorporated by reference for all purposes.

### FIELD

Embodiments of the subject matter disclosed herein relate to patient care protocol management, and more particularly, to integrated alert management for patient care.

### BACKGROUND

In general, theranostics describes combining therapeutics and diagnostics into one treatment protocol to treat a variety of cancers, including prostate, breast, thyroid, bone, neuroendocrine, and others. Theranostics protocols may include chemotherapy, gene therapy, radiation, and more. Therapeutic treatment is combined with diagnostics to allow for imaging, diagnosing, treatment of disease, and monitoring of response to treatment all at the same time. As a use-case example, a molecule called prostate-specific membrane antigen (PMSA) is highly expressed in a majority of prostate cancers, and as such it is a strong biomarker for the disease. By linking a small radiotracer to a targeted biomarker, imaging and treatment of the cancer can be performed together. However, theranostics protocols, such as those used to treat PMSA-positive prostate cancers, can often be timeline specific and correlating multiple steps of each cycle of treatment can be challenging when there are a multitude of EMRs, lab systems, scheduling systems, and the like.

### BRIEF DESCRIPTION

In one example, a computing device comprising a display screen, the computing device being configured to display on the display screen a menu listing one or more electronic medical records (EMRs) of one or more patients, and additionally being configured to display on the display screen a theranostics graphical user interface (GUI) accessible from the menu, wherein the theranostics GUI displays, for each patient, a pathway tracker indicating status of a corresponding theranostics protocol, wherein the pathway tracker comprises, for each cycle of the corresponding theranostics protocol, a limited snapshot of statuses of steps thereof, wherein each limited snapshot is selectable to launch a pop-up GUI with additional information relating to each of the steps thereof, and wherein the theranostics GUI is displayed while the one or more EMRs are in an un-launched state.

In another example, a method for a theranostics system comprises displaying a menu listing one or more options for retrieving data of a plurality of patients from a plurality of data repositories of a hospital system, the plurality of data repositories including one or more electronic medical record (EMR) systems, displaying a theranostics graphical user interface (GUI) that displays, for one or more of the plurality of patients, a pathway tracker indicating a first status of a current cycle of a theranostics protocol determined from the retrieved data from the one or more EMR systems, wherein the pathway tracker includes a snapshot element for each cycle of the theranostics protocol; and in response to a user selection of the snapshot element for a selected cycle, adding additional display elements to the theranostics GUI, the additional display elements identifying a second status of each step of the selected cycle determined from the retrieved data from the one or more EMR systems, wherein the theranostics GUI is displayed while the one or more EMR systems are in an un-launched state.

In another example, a theranostics system comprises one or more processors; and memory storing instructions executable by the one or more processors to: receive, at the theranostics system, a feed from an electronic medical record (EMR) database, where one or more patient parameters for each of a plurality of patients are sent over the feed; identify one or more patients of the plurality of patients associated with a theranostics protocol based on the received feed; output, for display on a display device, a theranostics graphical user interface (GUI) that includes, for the one or more patients of the plurality of patients, a pathway tracker comprising a plurality of cycle elements, wherein each of the plurality of cycle elements is selectable and displays a snapshot of steps of a given cycle, wherein each snapshot of steps is generated by applying a set of rules specific to the theranostics protocol to a set of patient data obtained from the feed; and display, on the theranostics GUI, for a selected patient of the one or more patients, a risk element that indicates whether a current cycle of the theranostics protocol is at risk.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an example computing system.

FIG. 2 shows a block diagram of an example module architecture for theranostics management.

FIG. 3 shows an example theranostics graphical user interface (GUI).

FIG. 4 shows a first pop-up GUI of a therapy date editor.

FIG. 5 shows a second pop-up GUI of a pathway tracker.

FIG. 6A shows a third pop-up GUI of a first patient summary.

FIG. 6B shows a fourth pop-up GUI of a second patient summary.

FIG. 7 shows a fifth pop-up GUI of an alert.

FIG. 8 shows a sixth pop-up GUI of the alert.

FIG. 9 shows a seventh pop-up GUI of a flag.

FIG. 10 shows an eighth pop-up GUI of the flag.

FIG. 11 shows a second example theranostics GUI.

FIG. 12 shows a third example theranostics GUI.

FIG. 13 shows a ninth pop-up GUI of an alert.

FIG. 14 shows a flowchart illustrating a method for receiving and analyzing data to display in a theranostics GUI.

FIG. 15 shows a flowchart illustrating a method for viewing and modifying a theranostics GUI.

### DETAILED DESCRIPTION

The following description relates to various embodiments of a theranostics management system. In particular, systems and methods for a theranostics management system including a pathway tracker are provided. Hospitals and outpatient clinical medical facilities may provide computing systems with graphical user interfaces (GUIs) for displaying patient information to healthcare providers and other users. In this way, a healthcare provider may view the most up-to-date patient information and retrieve data from electronic medical records (EMRs), imaging results, laboratory results, and so on. Further alerts may be automatically and/or manually generated to indicate status(es) of one or more steps of a theranostics protocol to which a patient is connected.

Theranostics describes combining therapeutics and diagnostics into one treatment protocol to treat a variety of cancers, including prostate, breast, thyroid, bone, neuroendocrine, and others. Theranostics protocols may include chemotherapy, gene therapy, radiation, and more. Therapeutic treatment is combined with diagnostics to allow for imaging, diagnosing, treatment of disease, and monitoring of response to treatment all at the same time. In some cases, a theranostics protocol may demand specific timeframes for administration of therapy. For example, a multi-cycle treatment protocol may include cycles that are spaced precisely apart at intervals such as 4 weeks, 6 weeks, 8 weeks, etc. Administration of therapy may also be dependent on patient status, including various lab results, vital signs, and the like, as well as a specific timeline for the drug itself (e.g., time between manufacturing, delivery, and administration). Theranostics protocols may include a multitude of steps, including acquisition of labs, visits with oncologists, delivery of corresponding drugs, imaging, and more. With multiple systems, including EMRs, external laboratory or diagnostic systems, and the like, providing information of a vast number of patients, coordination of theranostics protocols may be challenging. Healthcare providers may resort to manually monitoring and managing schedules for theranostics protocols for a plurality of patients in order to coordinate and track planned steps, delayed steps, and at risk steps. Manual management however becomes difficult when there are delays in doses, patients cancel appointments, or other disruptions occur.

The term “risk” as used in the present disclosure may indicate that a cycle or step of a cycle of a theranostics protocol is not on track to be completed because of an issue that may lead to the patient not being able to receive the medication as planned. For example, a lab abnormality indicating impaired renal function, cardiac abnormalities, or the like may lead a care provider to choose to not proceed with the dose administration. As another example, a delay in delivery of the medication for theranostics may lead to a patient not receiving the dose within an accepted timeframe. However, determination that a theranostics protocol or a cycle thereof is at risk may be delayed or otherwise difficult because care providers have to manually sort through lab results, schedules, and the like and monitor deliveries of medication with respect to scheduled therapy cycles, which is time consuming and error prone.

The methods and systems provided herein detail a theranostics management system in a single GUI that enables users to easily review patient status, a pathway tracker detailing status of each step in a given cycle, and any alerts determined therefor. The systems and methods may be configured to ingest data from a plurality of sources, including connected EMR(s), laboratory systems, and imaging systems as well as extract data from third parties (e.g., third party labs or imaging facilities) where result documents are scanned into the connected EMR(s). The systems may be configured to determine candidates for theranostics protocol, monitor patients assigned to a theranostics protocol, and track steps of each cycle of the theranostics protocol to determine any abnormalities. If any abnormalities are detected in labs, imaging, or otherwise, the system may display an alert and/or flag within the GUI. In this way, care providers may more efficiently and accurately manage patients associated with theranostics protocols.

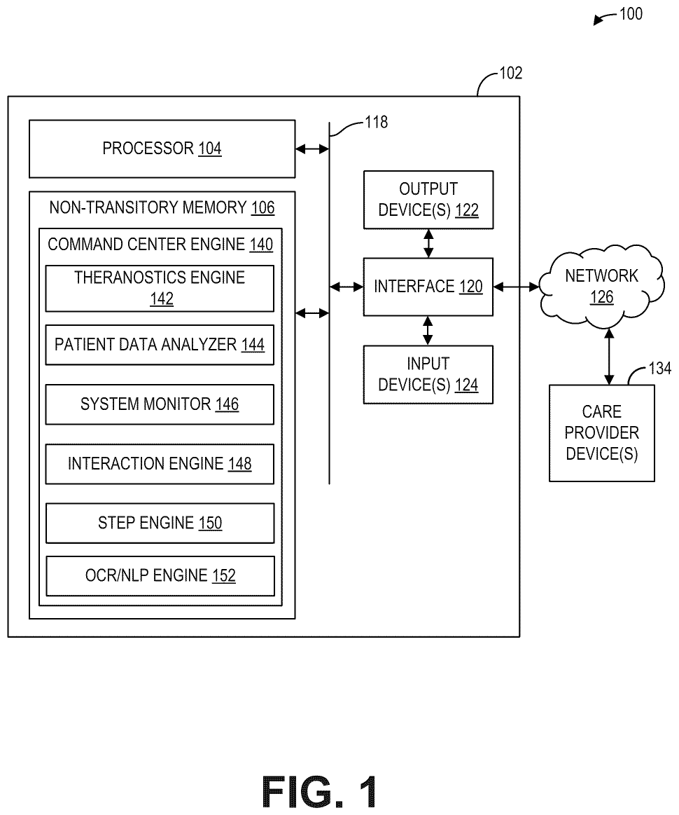

Referring now to FIG. 1, an example of a computing system 100 is shown. Computing system 100 comprises a computing device 102 which may comprise, as illustrative and non-limiting examples, a server, a personal computer, a workstation, a mobile device (e.g., a cellular phone, a smart phone, a computing tablet, and so on), or any other type of computing device.

The computing device 102 includes a processor 104 which may be configured to execute machine-readable instructions stored in non-transitory memory 106. Processor 104 may be single core or multi-core, and the programs executed thereon may be configured for parallel or distributed processing. In some embodiments, the processor 104 may optionally include individual hardware components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration. The computing device 102 further includes non-transitory memory 106. It should be appreciated that the computing device 102 may include additional memory devices, including volatile memory, mass storage, local memory, and so on.

In some examples, the non-transitory memory 106 may include components disposed at two or more devices, which may be remotely located and/or configured for coordinated processing. In some embodiments, one or more aspects of the non-transitory memory 106 may include remotely-accessible networked storage devices configured in a cloud computing configuration. The processor 104 and the non-transitory memory 106 may be coupled, for example, via a communications bus 118. As an example, the theranostics management system 100 may be integrated into a hospital environment that utilizes a single central server connected to a plurality of remote-access care provider devices (e.g., monitors), whereby each care provider device can access the theranostics management system 100 via the central server.

The computing device 102 may further include an interface 120 communicatively coupled to the processor 104 and the non-transitory memory 106 via the communications bus 118. The interface 120 may be implemented by one or more of any type of interface standard, such as an Ethernet interface, a universal serial bus (USB), a BLUETOOTH interface, a near field communication (NFC) interface, and/or a PCI express interface.

The computing device 102 may further include one or more output device(s) 122 communicatively coupled to the processor 104 and the non-transitory memory 106 via the interface 120. The output device(s) 122 may comprise, for example, one or more display devices. Such a display device may include one or more display devices utilizing virtually any type of technology (e.g., a light emitting diode (LED), an organic light emitting diode (OLED), a liquid crystal display (LCD), a cathode ray tube (CRT) display, an in-place switching (IPS) display, a touchscreen, and so on). In some examples, output device 122 may comprise a computer monitor configured to display medical information of various types and styles. Output device(s) 122 may be combined with processor 104, non-transitory memory 106, and/or user input device(s) 124 in a shared enclosure, or may be a peripheral display device and may comprise a monitor, touchscreen, projector, or other output device known in the art, which may enable a user to view decision support output (e.g., alerts) according to one or more examples of the current disclosure, and/or interact with various data stored in non-transitory memory 106.

The computing device 102 may further include one or more user input device(s) 124 coupled to the processor 104 and the non-transitory memory 106 via the interface 120. A user input device 124 may comprise, for example, one or more of a touchscreen, a keyboard, a mouse, a trackpad, a motion sensing camera, a microphone, or other device configured to enable a user to interact with and manipulate data within computing device 102.

The interface 120 may further include a communication device such as a transmitter, a receiver, a transceiver, a modem, a residential gateway, a wireless access point, and/or a network interface to facilitate exchange of data with external machines (e.g., computing devices of any kind) via a network 126. For example, the communication may be via, for example, an Ethernet connection, a digital subscriber line (DSL) connection, a telephone line connection, a coaxial cable system, a satellite system, a line-of-site wireless system, a cellular telephone system, and so on. As a non-limiting example, FIG. 1 shows one or more care provider devices 134 that may be communicatively coupled to computing device 102. Each care provider device may include a processor, memory, communication module, user input device, display (e.g., screen or monitor), and/or other subsystems (similar to the processor, memory, communication module, user input device, and output device of computing device 102) and may be in the form of a desktop computing device, a laptop computing device, a tablet, a smart phone, or other device. Each care provider device may be adapted to send and receive encrypted data and display medical information, including medical images in a suitable format such as digital imaging and communications in medicine (DICOM) or other standards. As will be explained in more detail below, the care provider devices may display graphical user interfaces described herein (including alerts generated by a theranostics engine 142) and may facilitate user interaction with the graphical user interfaces (e.g., to snooze, dismiss, or perform other actions for an alert).

The command center engine 140 may comprise a theranostics management system configured to enable evolving statuses and decision support associated with theranostics protocols. The command center engine 140 includes, as an illustrative and non-limiting example, a theranostics engine 142, a patient data analyzer 144, a system monitor 146, an interaction engine 148, a step engine 150, and an optical character recognition (OCP)/natural language processing (NLP) engine 152. The patient data analyzer 144 processes available patient data for patients of a healthcare facility to understand the patient population. For example, the patient data analyzer 144 tracks patients, associates them as candidates or non-candidates for one or more theranostics protocols, and analyzes laboratory, imaging, and other data of the patients. The step engine 150 integrates data from the patient data analyzer 144 in order to determine status of each of a plurality of steps of one or more cycles of an associated theranostics protocol. For example, each cycle of a given theranostics protocol may comprise a pre-therapy lab step, a pre-therapy oncology visit step, a therapy step, a post-therapy imaging step, a post-therapy lab step, and a post-therapy oncology visit step, as a non-limiting example. The step engine 150 may integrate data from the patient data analyzer 144 to determine status of each step, such as completed, scheduled, unscheduled, delayed, on track, at risk, etc. The system monitor 146 monitors a total resource load on the healthcare facility from patients and pathways and draws information from the patient data analyzer 144 and the step engine 150 to monitor patient progress with regard to individual step status and cycle status based on the individual steps statuses, and evaluate which patients' theranostics protocols are at risk and which are on target.

The interaction engine 148 enables one or more users and/or external systems (e.g., an electronic medical record system, a picture archiving and communication system, a radiology desktop, an imaging workstation, an administrative workstation, and so on) to interact with patient and/or theranostics protocol information via the system monitor 146, step engine 150, patient data analyzer 144, and the theranostics engine 142. For example, changes can be made to patient records, prescriptions, associated protocols/tasks, and so on, via the interaction engine 148. A display driver may generate a graphical user interface to display information on an output device 122 such as a display screen, for example, and facilitate interaction with the interaction engine 148.

The OCP/NLP engine 152 may analyze and extract data from third party laboratory or imaging results. For example, third party laboratory or imaging results may be stored in the system as a portable document format (PDF) or other type of document. The OCP/NLP engine 152 may analyze the PDF to extract characters (e.g., letters, numbers, and punctuation) and reformat the information in the PDF to text. The reformatted text may then be analyzed to extract data usable by the command center engine 140, such as lab values and dates thereof.

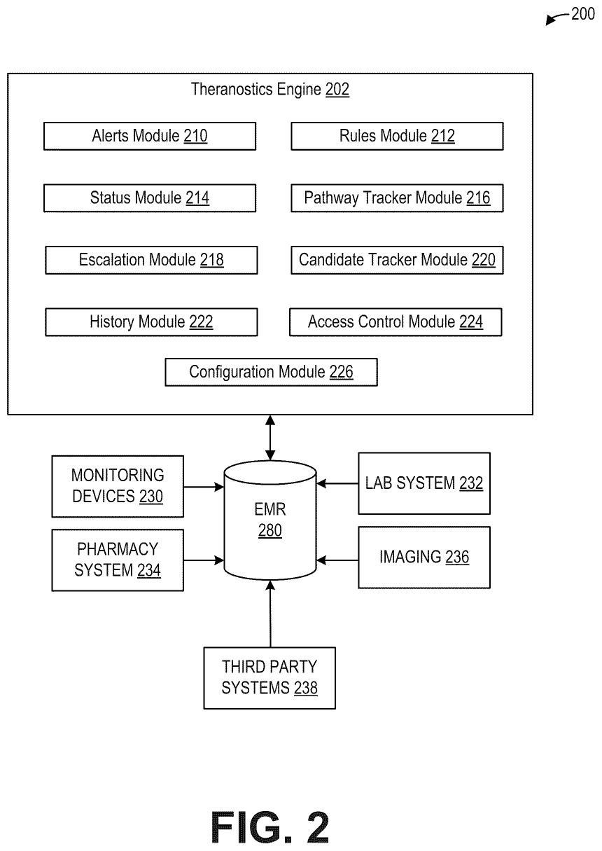

The theranostics engine 142 integrates into a graphical user interface or graphical user interface module (e.g., a tile) for enabling a theranostics protocol pathway tracker. As described further herein with regard to FIG. 2, the theranostics engine 142 provides a pathway tracker and associated alerts relating to a patient on a theranostics protocol responsive EMR signals and/or manual input, and further enables management of the alerts by selectively allowing a user to change a status of the alert, snooze or dismiss the alert, escalate the alert, comment on the alert, view history of the alert and actions relating to the alert, delete the alert, and so on.

The step engine 150 looks at a series of alerts from the theranostics engine 142, for example, for a patient and determines which steps of a theranostics protocol the patient has completed, which are scheduled, which are delayed, and which are not yet scheduled. For example, the patient may have a most recent creatinine level of greater than 2, which, on its own or in combination with other criteria, may trigger an alert for consideration by a care provider as to the patient's status for proceeding with a current cycle. As an illustrative example, the step engine 150 looks at pre-therapy labs drawn on a particular date. The step engine 150 includes a configuration table that includes a set of labs that are to be completed prior to therapy and if not scheduled and/or not yet completed, an alert may be triggered. Further, if the pre-therapy labs have been completed, the step engine 150 may integrate data from the patient data analyzer 144 to determine abnormalities in the pre-therapy labs, such as an elevated creatinine. Such abnormalities may also trigger alerts to be displayed in the GUI. If any alerts are triggered that indicate potential issues in completing the theranostics protocol are detected, including lab abnormalities, unscheduled steps, delays in dose transport or manufacture, and the like, an element indicating the step and the corresponding cycle are at risk may be displayed.

Determination and identification of alerts based on a plurality of patient parameters may stem from the configuration table and rule-based inputs from the electronic medical record (EMR) database. As an example, a patient currently undergoing theranostics treatment for prostate cancer with a radiopharmaceutical medication may be have six cycles of treatment planned, each cycle comprising a plurality of steps including labs, imaging, and medication administration. Data from EMRs, imaging systems, and third party data may be acquired and analyzed to determine schedule of steps and results to indicate any potential abnormalities or reasons for delay of treatment. In this way, theranostics protocol management may be automated without need for manual monitoring and management by a care provider. This in turn may reduce time and effort spent by the care provider in tracking and monitoring each patient on theranostics protocols.

It should be understood that computing system 100 shown in FIG. 1 is illustrative and non-limiting, and that another appropriate computing system 100 may include more, fewer, or different components.

FIG. 2 shows a block diagram of an example theranostics management system 200 for theranostics management. The theranostics management system 200 includes a theranostics engine 202 which may comprise the theranostics engine 142 of the command center engine 140, as an illustrative and non-limiting example. The theranostics engine 202 may include an alerts module 210, a rules module 212, a status module 214, a pathway tracker module 216, an escalation module 218, a candidate tracker module 220, a history module 222, an access control module 224, and a configuration module 226. The theranostics engine 202 may generate alert elements to be displayed within a GUI/tile.

The alerts module 210 generates alerts for patients responsive to automatic triggers (e.g., from EMR signals) and/or manual commands. Alerts may be displayed in the form of widget elements, such as current cycle risk elements. For example, the rules module 212 stores rules for evaluating EMR signals to determine alerts based on the EMR signals, determine if alerts are resolved based on EMR signals, and/or determine if conflicts or mismatches exist between alerts. The status module 214 manages a status of a step, for example scheduled, completed, delayed, in-progress, pending (e.g., pending collection, pending results, etc.), abnormal result, and the like, as may be determined for each step in each cycle of a patient's associated theranostics protocol. The status module 214 may, in some examples, further manage a priority of the alert.

The pathway tracker module 216, in conjunction with the alerts module 210 and the status module 214, may generate a pathway tracker for a theranostics protocol for a corresponding patient. The tracker may include a snapshot of step of each cycle of the theranostics protocol and any alerts and statuses thereof. The tracker may detail alerts that contribute to a risk alert for a cycle, in some examples. The escalation module 218 enables a user to escalate an alert to one or more users or departments. The candidate tracker module 220 may allow a user to track a candidate patient for a theranostics protocol.

The theranostics engine 202 operates globally across all users of the command center engine/theranostics engine. For example, when new lab results are obtained, all users will see indication of completed labs in the tracker and any abnormality alerts corresponding thereto in the GUI. This applies for all actions taken by the command center engine. In some examples, the command center engine may have an edit feature provided via access control module 224 that enables permissions/actions for each alert to be controlled at the user, group, or core level. For example, permissions for any action may be managed at the user group level so that a local team can adjust a specific alert or alert type to meet their specific implementation.

The access control module 224 controls access to different functions of the theranostics engine 202. Access to alert manager functions may be defined at a tile by tile level. The level of access may be at the following levels: User Can Snooze (Y/N), User Can Dismiss (Y/N), User Can Reactivate (Y/N), User Can Manage (Y/N), User Can Mark In Progress (Y/N), User Can Assign (Anyone, Self Only, No), User Can Configure Tile (Y/N), User Can Mark Priority (Y/N), and the like.

The history module 222 logs any and all action taken by a user regarding an alert or a comment. This history may be maintained such that it can be displayed on the tile and audited afterwards by authorized users. The information includes user ID, time of event, action taken, and any reason comments associated.

The configuration module 226 allows for customization of prioritization of alerts and customization of alert triggers, including lab triggers, schedule triggers, and the like. In some examples, care providers may configure a theranostics protocol and may configure which abnormalities can be detected to trigger alerts, including abnormalities or scheduling issues that may trigger an at risk alert for a cycle.

The configuration module 226 may further allow for customization of thresholds of criteria. As an example, a post-therapy imaging step may be unscheduled but depending on threshold dates configured, the post-therapy imaging step, when unscheduled, may or may not trigger an alert. For example, if unscheduled within a week of the date of dose administration, the unscheduled post-therapy imaging step may not trigger an alert, but if unscheduled more than a week out from the date of dose administration, the unscheduled post-therapy imaging step may trigger an alert. Thresholds may also be configured for lab values and other scheduling parameters in the system, in some examples.

As explained above, the alerts module 210 generates alerts for patients responsive to automatic triggers, which may include triggers from EMR signals. As such, the theranostics engine 202 is communicatively coupled to one or more EMR databases 280 such that the EMR databases 280 may transmit EMR signals (e.g., medical data) over a feed to the theranostics engine 202. EMR databases 280 may store EMRs for a plurality of patients. An EMR for a patient may include patient demographic information, family medical history, past medical history, lifestyle information, comorbidities (e.g., preexisting medical conditions), current medications, allergies, surgical history, past medical screenings and procedures, past hospitalizations and visits, etc. EMR databases 280 may be external databases accessible via a secured hospital interface, and/or EMR databases 280 may be local databases (e.g., housed on a device of the hospital). EMR databases 280 may be databases stored in a mass storage device configured to communicate with secure channels (e.g., HTTPS and TLS), and store data in encrypted form. Further, the EMR databases may be configured to control access to patient electronic medical records such that only authorized healthcare providers may edit and access the electronic medical records. In some examples, more than one EMR database may be communicatively coupled to the theranostics engine 202, for example if multiple EMR systems are used by or communicate with a facility. The EMR databases 280 may be data repositories from which a plurality of patient parameters may be received.

Further, theranostics engine 202 may communicate with other data sources that may supply triggers for generating alerts, such as lab system 232, pharmacy system 234, one or more monitoring devices 230, imaging services 236, and third party systems 238. The third party system 238 may include third party lab systems, third party imaging systems, and/or the like that may acquire data separate from the EMR database 280. As previously described, data acquired by third party systems may be stored in the EMR database 280 as PDF files, in some example, which may be analyzed and processed to reformat the data into a usable format by the theranostics engine 202, for example by the OCR/NLP engine 152 of FIG. 1. Other systems may be communicatively coupled to the theranostics engine 202 (directly and/or through EMR database), such as a computerized provider order entry (CPOE) system (which may track/manage provider orders such as treatments, tests, hospital ward/unit assignment changes, and the like) and the like.

The lab system 232 may include one or more computing devices associated with an on-site or off-site laboratory that performs lab tests on patient specimens. The one or more computing devices may include resources (e.g., memory and processors) allocated to store and execute a laboratory information system (LIS). The LIS may manage various aspects of the laboratory procedures, such as managing/assisting with tagging of incoming specimens (e.g., with patient and care provider information, test(s) to be conducted on the specimen, and so forth), tracking specimens (e.g., in storage, being processed), generating reports of test results, and the like. Accordingly, the LIS may interface directly with various laboratory equipment, such as mass spectrometers, chromatographers, analyzers, etc., and thus may have knowledge of which specimens are currently being tested, the results of such tests, and the performance status of the various pieces of equipment. The lab system 232 may send lab results to EMR database 280, and the lab results may be included in the EMR signals. Additionally or alternatively, the lab system 232 may send lab results directly to theranostics engine 202, at least in some examples, and thus the theranostics engine 202 may receive lab signals from which alerts may be generated.

The pharmacy system 234 may include one or more computing devices associated with an on-site or off-site pharmacy that fills prescriptions as ordered by care provider(s). The one or more computing devices of the pharmacy system may include resources (e.g., memory and processors) allocated to receive prescription requests and communicate the requests with pharmacy staff, track prescription fill status, notify an ordering care provider when a prescription is available, and so forth. The pharmacy system 234 may send prescription notifications to EMR database 280, and the prescription notifications may be included in the EMR signals. Additionally or alternatively, the pharmacy system 234 may send notifications regarding prescriptions directly to theranostics engine 202, at least in some examples, and thus the theranostics engine 202 may receive pharmacy signals from which alerts may be generated.

The monitoring devices 230 may include traditional medical devices monitoring respective patients, such as pulse oximeters, heart rate monitors, blood glucose monitors, and electrocardiograms (ECGs), as well as microphones, cameras, and other devices. The monitoring devices 230 may send output directly to the theranostics engine 202 and/or may send output to the EMR database 280. The imaging services 236 may include a radiology information system (RIS), a picture archive and communication system (PACS), and/or other computing devices associated with diagnostic imaging. The computing devices comprising the imaging services 236 may manage scheduling of diagnostic imaging exams, recommend and execute scanning protocols to perform diagnostic imaging exams, process imaging data to generate images, save diagnostic images in memory, etc. The imaging services 236 may send information/signals regarding diagnostic imaging to the EMR database 280 and/or directly to the theranostics engine 202.

In this way, the theranostics system herein presented is implemented using a specialized computing architecture configured to process and analyze large volumes of medical data in real-time. In particular, the architecture includes a distributed database system optimized for storing and retrieving heterogeneous medical data from multiple sources. This system utilizes a custom indexing scheme that allows for rapid retrieval of patient data based on complex queries involving multiple parameters such as diagnosis codes, lab values, and treatment protocols. Further, the theranostics management system includes a high-performance data ingestion pipeline that can process incoming data streams from various medical systems (e.g., EMRs, lab systems, imaging systems, etc.) at relatively high rates (e.g., exceeding 10 records per second). This pipeline employs parallel processing techniques and custom data parsing algorithms to extract relevant information from diverse data formats.

Further still, the theranostics management system includes a real-time alerting engine that can process complex rule sets defining theranostics protocols and generate alerts within milliseconds of detecting a relevant event or data point. This engine uses an optimized in-memory rules processing algorithm, as described above, that can evaluate thousands of rules per second for each patient.

Additionally, the theranostics management system may be configured to render the GUIs herein described, wherein the GUIs can dynamically generate and update complex visualizations of patient data and protocol status with minimum latency. For example, the system may utilize GPU acceleration and efficient data structures to maintain smooth performance even when displaying data for hundreds of patients simultaneously. Real-time updates to the GUI across multiple devices may be executed by a secure communication layer that maintains data security of the system. For example, the secure communication layer may employ an end-to-end encryption and token-based authentication to protect sensitive patient data.

These technical components work in concert to create a system that goes beyond mere data display or simple alert generation. The theranostics management system represents a significant technological improvement over existing medical information systems by enabling real-time, intelligent analysis and visualization of complex treatment protocols across large patient populations. This technological solution addresses the specific problem of managing intricate, time-sensitive theranostics protocols in a way that would be impractical or impossible using traditional methods or generic computing systems. In particular, as an example, with current computing systems, in order to retrieve and display disparate information, multiple EMRs may be accessed and individual reports, such as lab result reports (either within an EMR or rendered as a PDF) may be displayed on a display screen at the same time. In comparison, the theranostics management system aggregates this disparate information and renders a single GUI for display that includes all relevant information including abnormalities in labs, imaging, and scheduling disruptions. In this way, the theranostics management system increases overall processing efficiency of the computing device by decreasing the amount of data that is separately accessed and displayed.

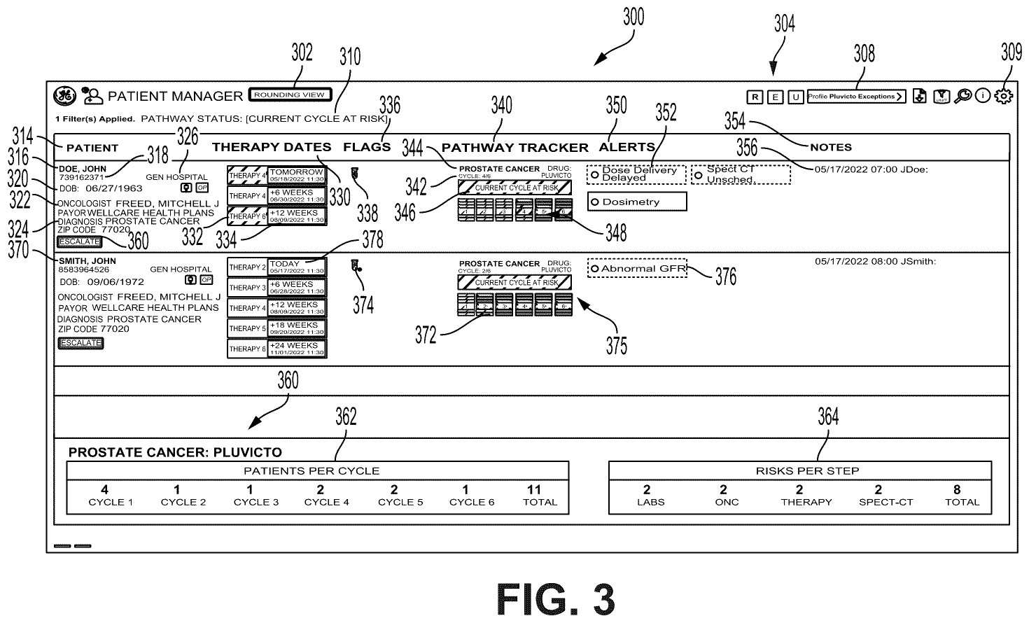

Turning to FIG. 3, an example theranostics GUI 300 is shown. The theranostics GUI 300 may be displayed on a display device (e.g., a display device of the care provider device 134) based on a theranostics engine, such as theranostics engine 202 of FIG. 2. Specifically, theranostics GUI 300 may be displayed to a care provider when the care provider uses the theranostics engine to view patient information for one or more patients of a medical facility that are currently associated with a theranostics protocol.

Theranostics GUI 300 may be viewed in one of a plurality of views, including a rounding view and an expediter view as non-limiting examples. As noted by current view element 302, the theranostics GUI 300 is depicted in FIG. 3 in rounding view. In some examples, different views may display information in different formats, such as fixed formats or customizable formats. The user may toggle between views by selecting one of a plurality of view elements 304. The selected one of the plurality of view elements 304 may corresponding to the current view element 302.

Theranostics GUI 300 may also corresponding to a selected profile within profile clement 308. Via the profile element 308, the user may select a desired profile for how information is displayed via the theranostics GUI 300. Various profiles may display different information within the GUI, including which theranostics protocol to designate, which patients are displayed, and what type of details about those patients is displayed. For example, an exceptions profile may include details of patients who are currently associated with a designated theranostics protocol while a candidates profile may include details of patients who are considered candidates for a theranostics protocol but are not actively associated with the protocol. Additionally, in some examples, one or more filters may be applied to the theranostics GUI 300 to filter patient data shown in the theranostics GUI 300. The one or more filters may be selected, for example, via controls accessible via a settings button 309. Filters selected may be displayed to the user via filter clement 310. For example, in the theranostics GUI 300 as shown in FIG. 3, a filter of pathway status is selected such that only patients with current cycles at risk are displayed within the GUI.

In the example shown in FIG. 3, scheduled therapy dates, flags, a pathway tracker, alerts, and notes are displayed in columns of the theranostics GUI 300, where each row corresponds to a patient associated with the designated theranostics protocol. In other examples, a layout of the information may be different. For example, in other embodiments, each patient may be displayed in a separate column and the details thereof may be displayed in rows. In some examples, which details are displayed in columns (or rows) may be configured by the user. It should be appreciated that the layout of the elements of a theranostics GUI may vary in different examples, and the elements may appear in different visual configurations without departing from the scope of this disclosure. Additionally, not all of the elements shown in theranostics GUI 300 may be included in an example, and some examples may include a greater or lesser number of elements.

Theranostics GUI 300 may include a patient information column 314. In some examples, the theranostics GUI 300 may display multiple patients at one time and each patient may comprise a separate row within the patient information column 314. In some examples, each row within the patient information column 314 may comprise a patient name 316, a medical record number 318, a birth date 320, a provider name 322, and a diagnosis 324, among other information, that identifies the patient to which the row corresponds. In other examples, other identifying information or less identifying information may be included in the patient information column 314. In some examples, each row of the patient information column 314 may additionally include a location 326 that identifies a location within a hospital facility that the corresponding patient is currently residing and/or where therapy for the associated theranostics protocol is to be administered. For example, for an outpatient theranostics protocol, an “OP” icon may be included in the location 326.

Theranostics GUI 300 may further include a therapy dates column 330, wherein each row of the therapy dates column 330 corresponds to a patient included in the theranostics GUI 300. Each row of the therapy dates column 330 may comprise planned therapy cycles and corresponding dates for which each planned therapy cycle is scheduled or anticipated to be scheduled. For example, a planned sixth therapy cycle element 332 may include a date panel 334 for which the planned sixth therapy cycle is scheduled or anticipated to be scheduled. In some examples, each planned therapy cycle displayed within a row of the therapy dates column 330 may be displayed with a color corresponding to its status. The status of each planned therapy cycle may be either “on target”, displayed as a first color such as green, or “at risk”, displayed as a second color such as red. Planned therapy cycles that are on target may include steps without issues (e.g., no unscheduled steps, no anticipated delays, etc.). Conversely, planned therapy cycles that are at risk may have one or more steps with issues (e.g., unscheduled steps, anticipated delays, incomplete labs, lab abnormalities, etc.). The planned therapy cycles and displayed colors thereof may be triggered based on EMR data, as determine by the theranostics engine of FIG. 2, according to a set of rules for the corresponding theranostics protocol. For example, the theranostics engine may determine an abnormal lab result is present in pre-therapy labs for a fourth therapy cycle and therefore, the fourth therapy cycle element displayed within the therapy dates column 330 may be displayed as the second color (e.g., red) indicating that there is an issue with a step that puts the fourth therapy cycle at risk.

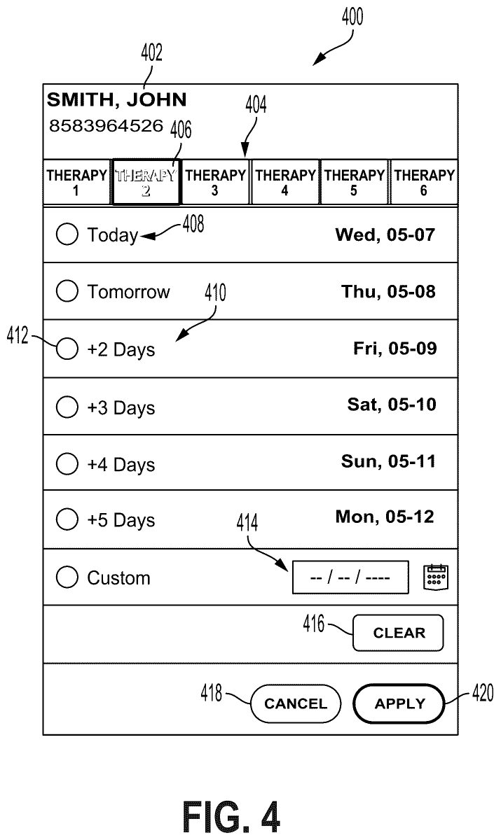

Further, in some examples, each of the displayed planned therapy cycles within the therapy dates column 330 may be selectable elements that, when selected, modify the theranostics GUI 300 to display a pop-up GUI (e.g., additional display panel). For example, the planned sixth therapy cycle element 332 may be a selectable element that, when selected, launches a pop-up GUI that allows the user to edit the date shown in date panel 334 for which the sixth planned therapy cycle is scheduled, as will be further described with respect to FIG. 4.

Theranostics GUI 300 may further include a flags column 336, wherein each row of the flags column 336 corresponds to a patient included in the theranostics GUI 300. Each row of the flags column 336 may include one or more flags and/or icons corresponding to patient details, for example lab results, imaging results, visit notes, and the like. For example, a lab flag 338 may be included in the flags column 336. In some examples, each flag and/or icon displayed within the flags column 336 may indicate whether there is an abnormality detected within corresponding lab results. For example, the lab flag 338 may be a first type of lab result flag that indicates that no abnormalities are detected. In contrast, a second lab flag 374 corresponding to a second patient 370 of the theranostics GUI 300 may be a second type of lab result flag (e.g., displayed with a color, such as the second color (e.g., red)) to indicate that one or more abnormalities are detected within the corresponding lab results.

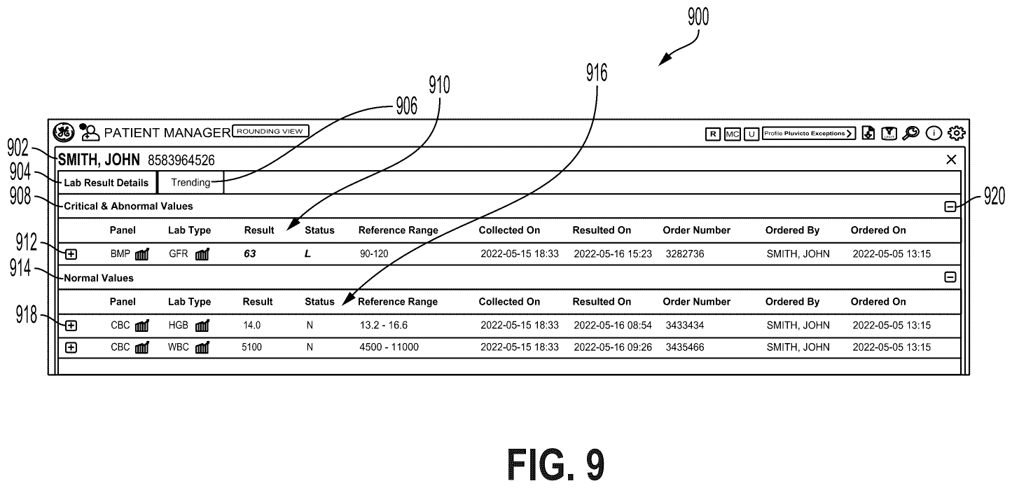

The flags and/or icons included in the flags column 336 may be selectable elements that, when selected via one or more types of user inputs, modify the theranostics GUI 300 to display pop-up GUIs (e.g., additional display panels) with additional information about the element that was selected. As an example, the second lab flag 374 may be selectable via a mouse click or hover, to display additional details of lab results, including details of the detected abnormalities, as will be further described with respect to FIG. 9.

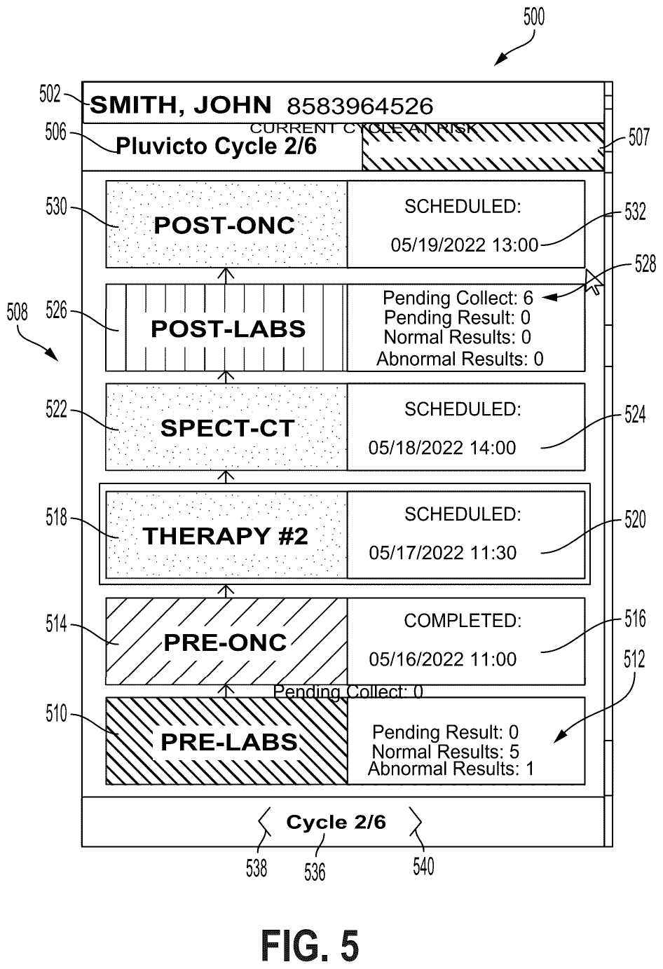

The theranostics GUI 300 may further include a pathway tracker column 340, wherein each row of the pathway tracker column 340 corresponds to a patient included in the theranostics GUI 300. Each row of the pathway tracker column 340 may comprise a diagnosis 344, a current cycle number 342, a risk element 346, and a cycle tracker 348. The risk element 346 may indicate whether the current cycle is at risk or not, in some examples via text, color, or a combination thereof. The current cycle may be at risk when one or more steps of the current cycle have an issue (e.g., are considered at risk). The cycle tracker 348 may comprise an element for each cycle of the designated theranostics protocol, including completed cycle(s), a current cycle, and planned future cycle(s). Each element of the cycle tracker 348 may include a limited snapshot of steps of the corresponding cycle, whereby each step is displayed as a color corresponding to status of the step. For example, steps displayed with the first color (e.g., green) may be on track, steps displayed with the second color (e.g., red) may be at risk due to some issue, such as a lab abnormality, being unscheduled, or an anticipated delay, steps displayed with a third color such as blue may be completed, and steps displayed with a fourth color, such as gray may be currently irrelevant (e.g., under a threshold timeframe from a current step or current date). Each element of the cycle tracker 348 may be selectable elements that, when selected, modify the theranostics GUI 300 by launching a pop-up GUI that includes additional information regarding the selected element. For example, a second cycle tracker 375 corresponding to the second patient 370 may include a cycle two element 372. The cycle two element 372 may be selectable to launch a pop-up GUI, as will be further described with respect to FIG. 5. In some examples, one or more of the cycle elements within the cycle tracker may correspond to therapy cycle elements within the therapy dates column 330. For example, the cycle two clement 372 may correspond to a planned second therapy date element 378.

Theranostics GUI 300 may further include an alerts column 350, wherein each row of the alerts column 350 corresponds to a patient included in the theranostics GUI 300. Each row of the alerts column 350 may include one or more alert elements, such as first alert element 352 in the first row and second alert element 376 in the second row. Alert elements in the alerts column 350 may correspond to determined issues or risks for the designated theranostics protocol. For example, the first alert element 352 indicates that dose delivery for the current cycle is delayed. Upon user selection of the first alert element 352, additional information about the alert, such as reason for dose delay, anticipated delivery time/date, and the like may be displayed. The second alert element 376 may indicate that an abnormal glomerular filtration rate (GFR) was detected in a patient's lab results. In some examples, alert elements in the alerts column 350 may correspond to cycle elements in the pathway tracker column 340 and/or flags/icons in the flags column 336. As an example, the second alert element 376 that indicates that an abnormal GFR was detected may correspond to the second lab flag 374, which also indicates that an abnormal lab result was detected.

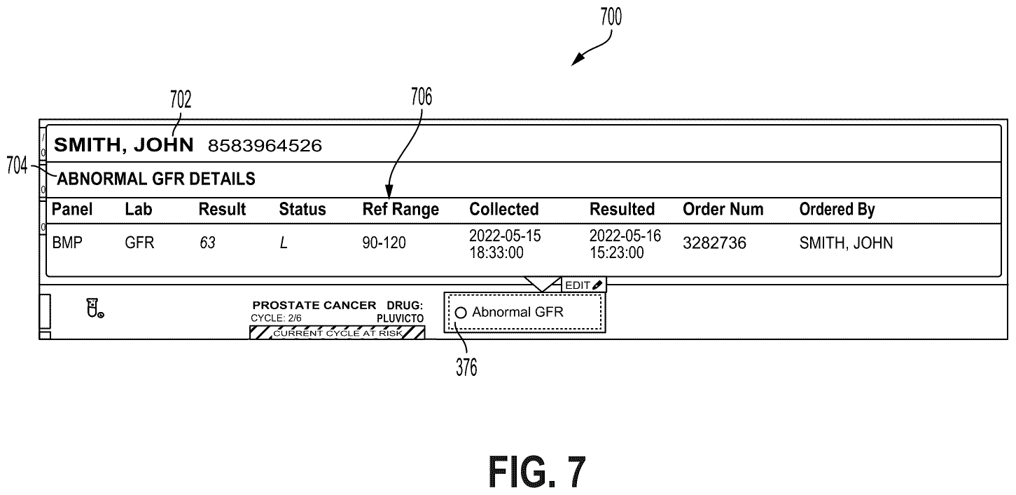

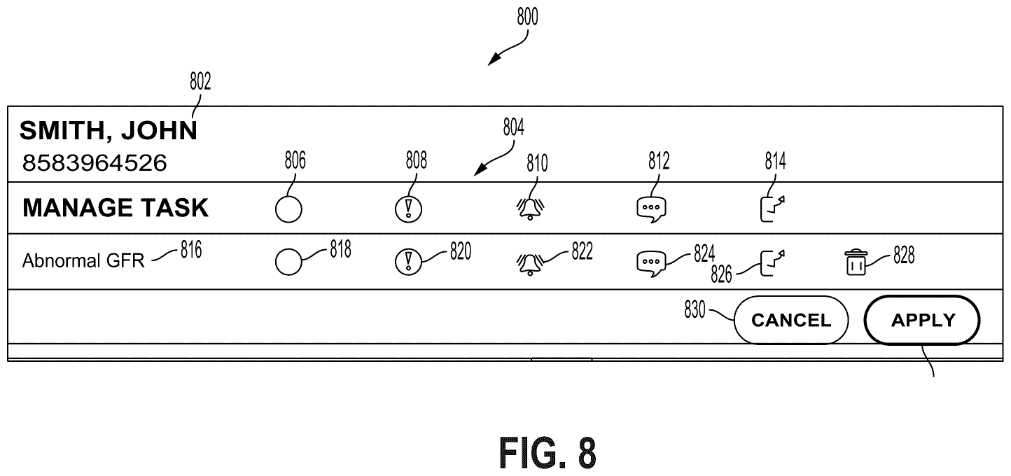

Similar to other elements in the theranostics GUI 300, alert elements in the alerts column 350 may be selectable elements that, when selected, modify the theranostics GUI 300 by launching a pop-up GUI. For example, the second alert element 376 may be selectable to launch a pop-up GUI that includes additional information about or editing of the abnormal GFR alert, as will be further described with respect to FIGS. 7-8. Different types of user inputs to some selectable elements such as the alert elements may modify the theranostics GUI 300 in different ways. For example, a hover over the second alert element 376 may launch a first panel including information about the alert, as will be described with respect to FIG. 7, while a mouse click of the second alert element 376 may launch a second panel that allows the user to edit or manage the alert, as will be described with respect to FIG. 8.

Theranostics GUI 300 may further include a notes column 354, wherein each row of the notes column 354 corresponds to a patient displayed within the theranostics GUI 300. Notes included in a row of the notes column 354 may correspond to an element elsewhere in the row. For example, note 356 may correspond to the first alert element 352, whereby the note 356 details a reason for dose delivery delay. Notes may be added to the notes column 354 via one or more pop-up GUIs, as will be further described below.

Theranostics GUI 300 may further include summary panels 360 that correspond to the designated theranostics protocol. The summary panels 360 may include a patients per cycle panel 362 and a risks per step panel 364. The patients per cycle panel 362 may include headings for each cycle of the designated theranostics protocol, wherein each heading details how many patients associated with the designated theranostics protocol are currently at that cycle. The risks per step panel 364 may include headings for each step of a cycle of the designated theranostics protocol, for example including labs (both pre and post-therapy), oncology visits (both pre and post therapy), therapy administration, and imaging. Each heading may detail how many patients associated with the designated theranostics protocol have issues or risks with the corresponding step. As described previously, issues or risks may include scheduling issues (e.g., unscheduled visits, uncollected labs, etc.), lab abnormalities, delays, dosage inconsistencies, and the like. Each heading in both the patients per cycle panel 362 and the risks per step panel 364 may be selectable elements that, when selected, launch pop-up GUIs or panels displaying additional information, including patient information, cycle information, and/or issue/risk information.

Turning now to FIG. 4, a first pop-up GUI 400 is shown. First pop-up GUI 400 may be a therapy date editor GUI and may be launched in response to the user selecting or hovering over a therapy cycle element within a therapy dates column of a theranostics GUI, for example the therapy dates column 336 of theranostics GUI 300. For example, first pop-up GUI 400 may be launched in response to user selection of the planned second therapy date element 378 of theranostics GUI 300. The first pop-up GUI 400 may therefore relate to the planned second therapy cycle to which the planned second therapy date element 378 corresponds. The first pop-up GUI 400 may be configured for the user to edit the date of the planned second therapy cycle. In some examples, the first pop-up GUI 400, and other pop-up GUIs herein described, may be displayed over the top of a theranostics GUI from which it is launched. In other examples, the first pop-up GUI 400 may be displayed as a separate window or as a side panel of the theranostics GUI from which it is launched.

The first pop-up GUI 400 may comprise a patient information heading 402. The patient information heading 402 may include a patient name and a medical record number. The information in the patient information heading 402 may correspond to the patient to which the element from which the first pop-up GUI 400 was launched corresponds to. The first pop-up GUI 400 may further comprise a plurality of therapy tabs 404. Each of the therapy tabs in the plurality of therapy tabs 404 may correspond to one of the cycles of the designated theranostics protocol. The first pop-up GUI 400 may be launched with a tab 406 of the plurality of therapy tabs 404 selected that corresponds to the planned therapy cycle from which the first pop-up GUI 400 was launched.

The first pop-up GUI 400 may comprise a potential dates column 408. The potential dates column 408 may comprise a plurality of dates that are selectable to schedule the planned therapy cycle. For example, potential date row 410 may be included in the potential dates column 408. The user may toggle between the plurality of therapy tabs 404 to change which planned therapy cycle is being managed. Each row in the potential dates column 408 may include a selectable element that when selected may indicate that the planned therapy cycle is to be scheduled for that date. For example, the potential date row 410 may include select element 412 that when selected assigns the date of the potential date row 410 to the planned therapy cycle. The potential dates column 408 may additionally include a custom date row 414 through which the user may select a date not elsewhere listed in the potential dates column 408. The user may clear a selected element by selecting a clear element 416. The user may exit the first pop-up GUI 400 without saving the selected date via a cancel element 418. The user may save the selected date by selecting an apply element 420.

Turning to FIG. 5, a second pop-up GUI 500 is shown. The second pop-up GUI 500 may be a pathway tracker GUI and may be launched in response to the user selecting or hovering over a cycle element within a pathway tracker column of a theranostics GUI, for example the pathway tracker column 340 of theranostics GUI 300. For example, second pop-up GUI 500 may be launched in response to user selection of the cycle two element 372 of the second cycle tracker 375 of theranostics GUI 300. The second pop-up GUI 500 may therefore relate to the second cycle of the theranostics protocol for the second patient. The second pop-up GUI 500 may be configured to display each step of the corresponding cycle and details thereof.

The second pop-up GUI 500 may comprise a patient information heading 502. The patient information heading 502 may include a patient name and a medical record number. The information in the patient information heading 502 may correspond to the patient to which the element from which the second pop-up GUI 500 was launched corresponds to. The second pop-up GUI 500 may further comprise a theranostics protocol name 506, which includes drug name and current cycle number. If the current cycle is at risk as is determined by detection of any abnormal labs, unscheduled visits or studies, anticipated delays, etc., an at risk element 507 may be displayed alongside the theranostics protocol name 506.

The second pop-up GUI 500 may comprise a plurality of step elements 508. The plurality of step elements 508 may depend on the designated theranostics protocol. In the example shown in FIG. 5, the designated theranostics protocol comprises a pre-therapy labs step, a pre-therapy oncology visit step, a therapy administration step, an imaging step, a post-therapy labs step, and a post-therapy oncology visit step. Therefore, the plurality of step elements 508 may comprise a pre-therapy labs step clement 510, a pre-therapy oncology visit step element 514, a therapy cycle step clement 518, an imaging step element 522, a post-therapy labs step element 526, and a post-therapy oncology visit step clement 530.

Each of the plurality of step elements may comprise an information panel. For example, the pre-therapy labs step element 510 comprises a first information panel 512 that details information about the pre-therapy labs, including number of lab orders that are pending collection, number of collected that are pending results, number of normal lab results, and number of abnormal lab results. Similarly, the post-therapy labs step clement 526 may comprise a second information panel 528 that details information about the post-therapy labs. The pre-therapy oncology visit step clement 514 may comprise a third information panel 516 that details a date of a scheduled pre-therapy oncology visit. Similarly, the post-therapy oncology visit step element 530 may comprise a fourth information panel 532 that details a date of a scheduled post-therapy oncology visit. The therapy cycle step clement 518 may comprise a fifth information panel 520 that details a scheduled date and time of the corresponding therapy administration. The imaging step clement 522 may comprise a sixth information panel 524 that details a scheduled date and time of the imaging exam. The imaging step clement 522 may also indicate in its title what the scheduled imaging exam is, for example a SPECT-CT, PET-CT, or the like.

Similar to as is described with respect to the theranostics GUI 300 of FIG. 3, each of the plurality of step elements 508 may be displayed in the second pop-up GUI 500 with a color that corresponds to status of the step. For example, step elements that correspond to steps that are on target (e.g., scheduled without anticipated issues or delays) may be displayed with the first color (e.g., green), including the therapy cycle step element 518, imaging step element 522, and post-therapy oncology visit step clement 530. Elements corresponding to steps that have issues or risks may be displayed with the second color (e.g., red), such as the pre-therapy labs step element 510 in which abnormal result(s) are included therein (as is displayed in the first information panel 512). The second color may therefore indicate an issue with the corresponding step that puts the current cycle at risk. Elements corresponding to completed steps may be displayed with the third color (e.g., blue), such as the pre-therapy oncology visit step element 514. Elements corresponding to steps that are not scheduled or completed but are not issues/risks for the current cycle may be displayed as the fourth color (e.g., gray). For example, the post-therapy labs step element 526 indicates in the information panel 528 that the post-therapy labs are pending collection. However, as the therapy cycle has yet to be completed, post-therapy labs inherently may not yet be collected and therefore uncollected post-therapy labs may not currently be an issue for the current cycle.

The second pop-up GUI 500 may further comprise a cycle toggle element 536. The cycle toggle element 536 may indicate which cycle is currently being displayed in the second pop-up GUI 500. The cycle toggle element 536 may comprise a preceding toggle element 538 and a succeeding toggle element 540. User selection of the preceding toggle element 538 may trigger display of step elements for a preceding cycle of the theranostics protocol for the corresponding patient. Similarly, user selection of the succeeding toggle element 540 may trigger display of step elements for a subsequent cycle of the theranostics protocol for the corresponding patient.

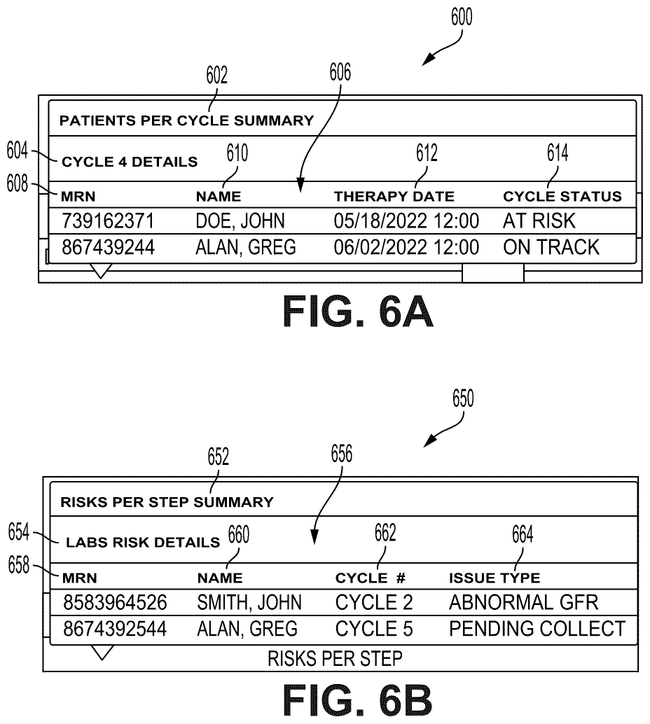

Turning now to FIGS. 6A and 6B, third and fourth pop-up GUIs of patient summaries are shown. FIG. 6A specifically shows a third pop-up GUI 600 and FIG. 6B specifically shows a fourth pop-up GUI 650. Both the third pop-up GUI 600 and the fourth pop-up GUI 650 may be launched in response to the user selecting or hovering over an element within a summary panel, such as one of the summary panels 360 of theranostics GUI 300. For example, third pop-up GUI 600 may be launched in response to user selection of an element of the patients per cycle panel 362 and fourth pop-up GUI 650 may be launched in response to user selection of an element of the risks per step panel 364.

The third pop-up GUI 600 may include a heading 602 indicating the patient summary that is displayed therein (e.g., patients per cycle summary) and a subheading 604 indicating which cycle is being summarized in the third pop-up GUI 600. The third pop-up GUI 600 may comprise a plurality of columns 606, wherein each identified patient of the patient summary is displayed in a row of the GUI. The plurality of columns may comprise a medical record number column 608, wherein the medical record number of each respective patient is shown, a name column 610, wherein the name of each respective patient is shown, a therapy date column 612, and a cycle status column 614. The therapy date column 612 may indicate a scheduled date and time or completion date and time for the cycle indicated by the subheading 604. The cycle status column 614 may indicate a status of the cycle for the respective patient, such as on track or at risk.

Similar to the third pop-up GUI 600, the fourth pop-up GUI 650 may include a heading 652 indicating the patient summary that is displayed therein (e.g., risks per step summary) and a subheading 654 indicating which step is being summarized in the fourth pop-up GUI 650. The fourth pop-up GUI 650 may comprise a plurality of columns 656, wherein each identified patient of the patient summary is displayed in a row of the GUI. The plurality of columns may comprise a medical record number column 658, wherein the medical record number of each respective patient is shown, a name column 660, wherein the name of each respective patient is shown, a cycle number column 662, and an issue type column 664. The cycle number column 662 may indicate which cycle the risk in the summarized step corresponds to. The issue type column 664 may indicate the issue with the step for each respective patient, for example an abnormal result or a pending collection for a lab step or a dose delivery delay for a therapy step.

Referring now to FIG. 7, a fifth pop-up GUI 700 is shown. The fifth pop-up GUI 700 may be an alert GUI and may be launched in response to a first type of user input, such as a hover, to an alert element within an alerts column of a theranostics GUI, for example the alerts column 350 of theranostics GUI 300. For example, fifth pop-up GUI 700 may be launched in response to user selection of the second alert element 376 of the alerts column 350. The fifth pop-up GUI 700 may therefore relate to the alert to which the second alert element 376 corresponds, in the depicted example the alert is an abnormal GFR result in a lab panel.

The fifth pop-up GUI 700 may comprise a patient information heading 702. The patient information heading 702 may include a patient name and a medical record number. The information in the patient information heading 702 may correspond to the patient to which the clement from which the fifth pop-up GUI 700 was launched corresponds to. The fifth pop-up GUI 700 may further comprise a subheading 704 that includes a description of the alert. The fifth pop-up GUI 700 may be configured to display additional information of the alert.

The fifth pop-up GUI 700 may include a plurality of columns 706. The plurality of columns may depend upon the alert to which the fifth pop-up GUI 700 corresponds. For example, for an abnormal lab result alert, as is depicted in FIG. 7, the plurality of column 706 may comprise a panel column, a lab name column, a result column, a status column, a reference range column, a collection date/time column, and a result date/time column. In other examples when other types of alerts are displayed within the fifth pop-up GUI 700, the columns may be different. For example, for a dose delivery delay alert, the columns may include a scheduled date/time column, a delay timeframe column, and a reason for delay column.

In FIG. 8, a sixth pop-up GUI 800 is shown. The sixth pop-up GUI 800 may be an alert management GUI. The sixth pop-up GUI 800 may be launched in response to a second type of user input, such as a mouse click, to an alert clement within a theranostics GUI. As a non-limiting example, the fifth pop-up GUI 700 may be displayed in response to hovering over the second alert element 376, as noted, while the sixth pop-up GUI 800 may be displayed in response to clicking on the second alert element 376. In comparison to the fifth pop-up GUI 700 which displays additional information of the alert, the sixth pop-up GUI 800 may be configured to allow the user to manage the alert. The sixth pop-up GUI 800 may comprise a patient information panel 802 that comprises a patient name and a medical record number, in some examples.

Managing the alert, as herein described, may comprise acknowledging the alert, escalating the alert, snoozing the alert, commenting on the alert, and deleting the alert. Thus, the sixth pop-up GUI 800 may comprise a plurality of management columns 804, each corresponding to one of the management options. For example, the plurality of management columns may include an acknowledgement column 806, a demands addressing column 808, a snooze column 810, a comment column 812, and an escalation column 814.

In some examples, while one alert element may be selected to launch the sixth pop-up GUI 800, all the alerts for the corresponding patient may be displayed within the sixth pop-up GUI 800. Each alert displayed within the sixth pop-up GUI 800 may comprise a row with an element for each column. For example, a row for an abnormal GFR alert 816 may comprise an acknowledgement element 818, an address element 820, a snooze element 822, a comment element 824, and an escalation clement 826. The sixth pop-up GUI 800 may further comprise a delete element 828.

The acknowledgement element 818, when selected via user input, may indicate to all users that the corresponding alert has been acknowledged, addressed, or completed. The demands addressing element 820, when selected via user input, may indicate that the alert is yet to be addressed. The snooze element 822, when selected via user input, may temporarily dismiss the alert for all users. The comment element 824, when selected via user input, may launch a comment window that allows the user to write a comment on the alert. In some examples, the comment, after being applied/saved, may then be displayed within a theranostics GUI for all users within a notes column. The escalation element 826, when selected via user input, may launch an additional window through which the user may escalate the alert to another user. The delete element 828, when selected via user input, may remove the alert. Any selections made in the sixth pop-up GUI 800 may be canceled via selection of a cancel element 830 or applied via selection of an apply element 832.

Referring to FIG. 9, an example of a seventh pop-up GUI 900 is shown. The seventh pop-up GUI 900 may be a lab flag GUI, in some examples. The seventh pop-up GUI 900 may be displayed in response to user selection of a lab flag or icon, for example the second lab flag 374 of FIG. 3. The information in the patient information heading 902 may correspond to the patient to which the element from which the seventh pop-up GUI 900 was launched corresponds to. The seventh pop-up GUI 900 may further comprise a plurality of result tabs, including a first tab 904 and a second tab 906. Each of the result tabs, when selected, may display information in the seventh pop-up GUI 900 in a different form.

The seventh pop-up GUI 900 is shown in FIG. 9 with the first tab 904 selected. When the first tab 904 is selected, the seventh pop-up GUI 900 may display an abnormal lab value panel 908 and a normal lab value panel 914. The abnormal lab value panel 908 may comprise a list 910 of each abnormal lab result for the corresponding patient from a specified lab draw, for example a pre-therapy lab draw for a current cycle. For example, the list 910 as shown in FIG. 9 depicts an abnormal GFR result in a first row. Each abnormal result may define a row of the list 910, and may include information such as panel type, lab type, result, status (e.g., low or high), reference range, collection date, result date, order number, ordering provider, and order date. Each row may additionally include an expansion button, such as expansion button 912. The expansion button, when selected via user input, may trigger expansion of the corresponding row so as to show a plurality of values for the corresponding lab type. For example, selection of the expansion button 912 may trigger display of every GFR result for the patient within a specified time range (e.g., 3 years) so the user may visualize a trend.

Similarly, the normal lab value panel 914 may comprise a list 916 of each normal lab result for the corresponding patient from the specified lab draw. For example, the list 916 as shown in FIG. 9 depicts a normal hemoglobin level in a first row. Each normal result may define a row of the list 916, and may include the same types of information as the abnormal lab value panel 908. Also similar to the abnormal lab value panel 908, each row of the list 916 may comprise an expansion button, such as expansion button 918 of the first row. The expansion button, when selected via user input, may trigger display of additional lab results of the lab type to which the row corresponds. Both of the panels may be collapsed via a collapse button, such as collapse button 920.

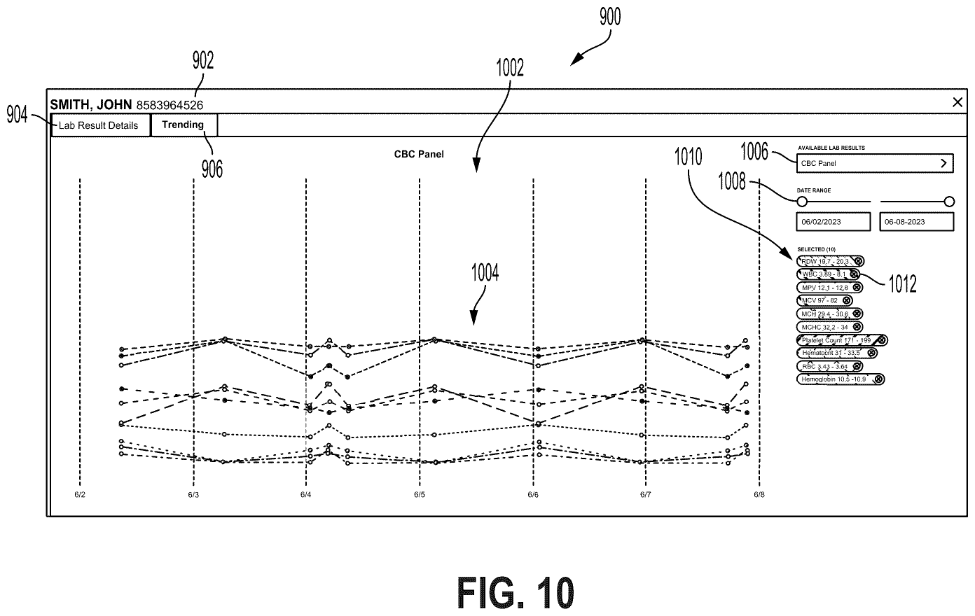

FIG. 10 shows the seventh pop-up GUI 900 when the second tab 906 is selected. When the second tab 906 is selected, the seventh pop-up GUI 900 may display a graph 1002. The graph 1002 may comprise a plurality of trend lines 1004, each corresponding to a particular lab value. For example, the graph 1002 may be specific to a CBC lab panel and each of the plurality of trend lines 1004 may correspond to a value of the CBC lab panel, such as white blood cell (WBC) count, platelet count, and the like. Which lab panel is viewed in the graph 1002 may be determined via a panel drop down menu 1006. A date range for the graph 1002 may be selected via a date range clement 1008. Selection of a lab panel from the panel drop down menu 1006 may trigger display of all lab value types included in the selected lab panel, in some examples. The seventh pop-up GUI 900 may comprise a plurality of lab value elements 1010 through which the user may refine which lab values are plotted on the graph 1002. For example, via a delete element 1012, the WBC values may be removed from the graph 1002. In this way, the user (e.g., care provider) may curate the graph 1002 to display the lab values they feel are most relevant, thereby reducing time searching the graph 1002 for desired values. The graph 1002 may display values for each desired lab value over the selected date range. In this way, the user may visualize trends of desired lab values over time to determine, as an example, whether an abnormality seen is a patient's baseline or a new change.

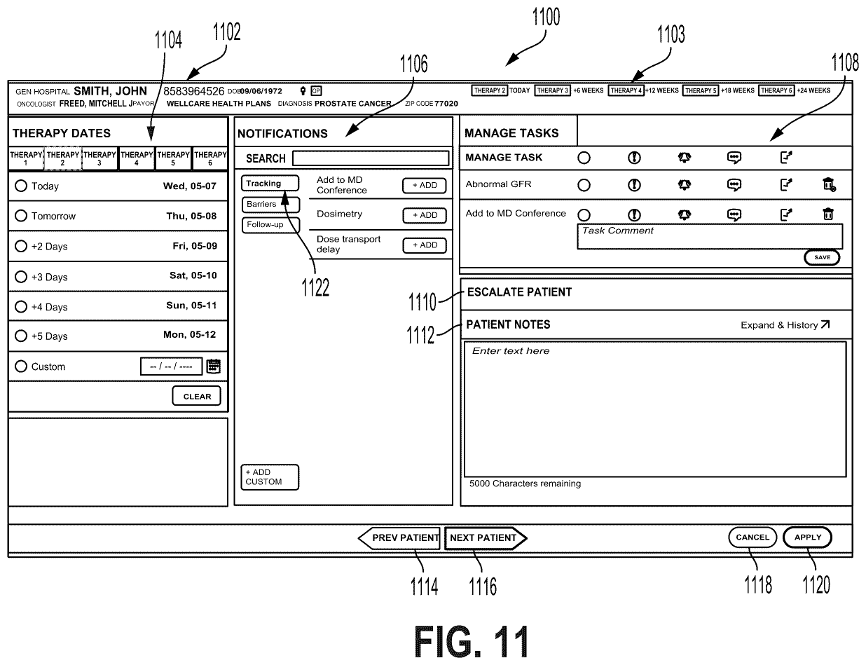

Referring now to FIG. 11, a theranostics GUI 1100 is shown. Theranostics GUI 1100 may be a patient editor GUI that comprises a plurality of other GUIs therein for managing patient care for a patient on a theranostics protocol. In some examples, the theranostics GUI 1100 may be launched in response to user selection of a patient, for example in response to selection of patient name 316 within theranostics GUI 300.

In some examples, the theranostics GUI 1100 may comprise a patient information panel 1102. The patient information panel 1102 may comprise a plurality of patient identifiers, including a name, a medical record number, a birth date, a location (e.g., outpatient), a care provider (e.g., oncologist), an insurance provider, a primary diagnosis (e.g., diagnosis for why they are on a theranostics protocol), and the like. The patient information panel 1102 may further comprise a list 1103 of anticipated therapy dates for each remaining cycle in the patient's theranostics protocol. In some examples, the anticipated therapy dates may be goal dates based on the theranostics protocol demands, but the therapy cycles may not be scheduled yet.

As noted, the theranostics GUI 1100 may comprise a plurality of other GUIs therein for managing patient care. The plurality of other GUIs may comprise a therapy date editor GUI 1104, a notifications GUI 1106, and an alert management GUI 1108 (e.g., task manager). The therapy date editor GUI 1104 may be similar to the first pop-up GUI 400 described with respect to FIG. 4. The therapy date editor GUI 1104 may allow the user to manage dates for therapy of each cycle, wherein the GUI comprises a plurality of potential dates each with an associated selection element that when selected indicates that the therapy for the indicated cycle is to be scheduled for that date.

The notifications GUI 1106 may allow the user to add alerts, tasks, and/or orders for a patient. For example, the notifications GUI 1106 may include a plurality of tabs 1122 that, when selected, triggers display of one or more suggested alerts, tasks, or orders. For example, a tracking tab, as is shown in FIG. 11, triggers display of one or more suggested alerts for tracking of the patient on the theranostics protocol, such as adding the patient to a conference schedule or alerting users to an anticipated delay in dose transport. A follow up tab may trigger display of one or more suggested follow up appointments, such as orders for a post-therapy oncology visit.

The alert management GUI 1108 may be similar to sixth pop-up GUI 800 as described with respect to FIG. 8. The alert management GUI 1108 may allow the user to manage one or more alerts or tasks assigned to the patient either manually, for example via the notifications GUI 1106, or automatically by the theranostics system. For example, the alert management GUI 1108 may include elements for acknowledging an alert, snoozing an alert, escalating an alert, commenting on an alert, and more.

The theranostics GUI 1100 may further comprise an escalate element 1110 that when selected escalates the patient to one or more other users. In contrast to the escalation element of the alert management GUI 1108 which escalates a specified alert or task, the escalate element 1110 may escalate the patient as a whole. The theranostics GUI 1100 may additionally comprise a patient notes element 1112 that includes a text box into which the user may enter notes (e.g., via typing on a keyboard of a care provider device). Patient notes entered may be displayed, for example, within the notes column 354 of theranostics GUI 300.

The theranostics GUI 1100 may comprise a previous patient button 1114 and a next patient button 1116 that when selected trigger display of a previous or next patient, respectively, if available. The list of patients through which the previous patient and next patient buttons 1114 and 1116 toggle may be based on a selected profile, for example all patients on a designated theranostics protocol or patients with cycles at risk on the designated theranostics protocol. A cancel element 1118 for canceling any selections made in the theranostics GUI 1100 and an apply clement 1120 for applying any selections made in the theranostics GUI 1100 may also be included. The theranostics GUI 1100 may as such concentrate patient care management into a single GUI, reducing time spent by the user in finding editing and/or management elements to manage alerts/tasks, add orders or new alerts, and change dates of therapies.

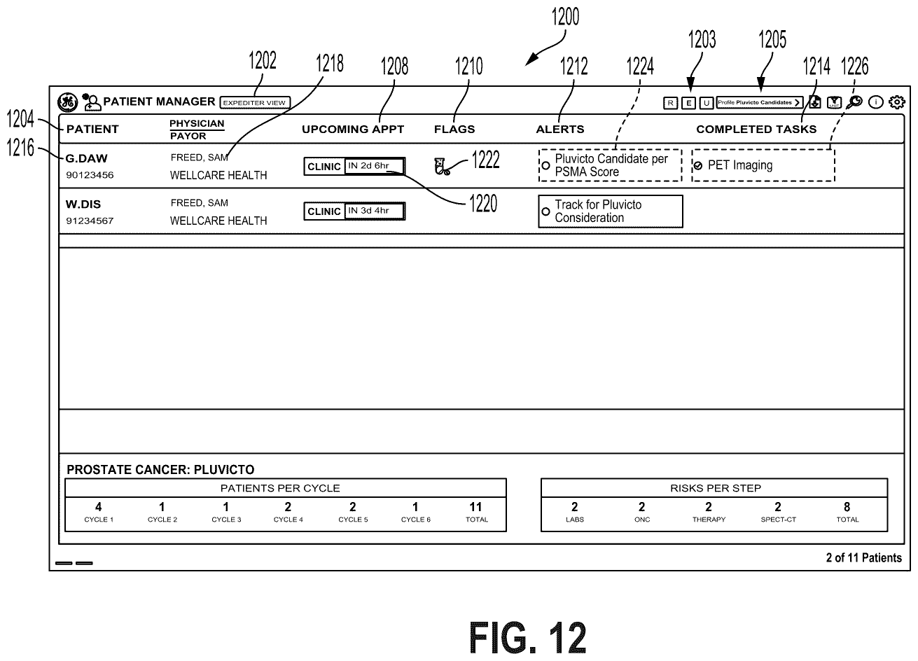

FIG. 12 shows another theranostics GUI 1200. The theranostics GUI 1200, in contrast to the theranostics GUI 300 of FIG. 3, displays information for patients who do not have current orders for a theranostics protocol but are identified by the theranostics system or manually by a care provider for a theranostics protocol. As an example, each patient displayed may have a diagnosis of prostate cancer and a positive PMSA, but may not have a current order for a radiopharmaceutical used to treat prostate cancer as part of a theranostics protocol.

Similar to the theranostics GUI 300, theranostics GUI 1200 may be viewed in one of a plurality of views, including a rounding view and an expediter view as non-limiting examples. As noted by current view element 1202, the theranostics GUI 1200 is depicted in FIG. 12 in expediter view. In some examples, different views may display information in different formats, such as fixed formats or customizable formats. The user may toggle between views by selecting one of a plurality of view elements 1203. The plurality of view elements 1203 may be the same elements as the plurality of view elements 304. The selected one of the plurality of view elements 1203 may corresponding to the current view element 1202.

Theranostics GUI 1200 may also correspond to a selected profile within profile element 1205. The profile element 1205 may be the same as the profile element 308 of theranostics GUI 300. When the profile element 1205 is toggled to an exceptions profile, all patients on a designated protocol with cycles at risk may be displayed, as is shown in FIG. 3. When the profile element 1205 is toggled to a candidates profile, all patients who are determined to be candidates for the designated theranostics protocol may be displayed

In the example shown in FIG. 12, upcoming appointments, flags, alerts, and completed tasks are displayed in columns of the theranostics GUI 1200, where each row corresponds to a patient who is a candidate for the designated theranostics protocol. In other examples, a layout of the information may be different. For example, in other embodiments, each patient may be displayed in a separate column and the details thereof may be displayed in rows. In some examples, which details are displayed in columns (or rows) may be configured by the user. It should be appreciated that the layout of the elements of a theranostics GUI may vary in different examples, and the elements may appear in different visual configurations without departing from the scope of this disclosure. Additionally, not all of the elements shown in theranostics GUI 1200 may be included in an example, and some examples may include a greater or lesser number of elements.

Theranostics GUI 1200 may include a patient information column 1204. In some examples, the theranostics GUI 1200 may display multiple patients at one time and each patient may comprise a separate row within the patient information column 1204. In some examples, each row, for example a first row, within the patient information column 1204 may comprise a patient name 1216 and a provider name 1218, however in other views (e.g., rounding view), additional patient information including medical record number, birth date, diagnosis, and more, may also be displayed within the patient information column 1204.

An upcoming appointment column 1208 may display a nearest upcoming appointment for each patient in a respective row. For example, an appointment clement 1220 corresponding to the patient of the first row. The appointment element 1220 may include a location (e.g., clinic, lab, imaging, etc.) and a date/time of the upcoming appointment. In some examples, the date/time may be displayed in terms of a current date/time (e.g., “IN 2 days and 6 hours) and in other examples, the date/time may be a calendar date and time.

A flags column 1210, similar to the flags column 336 of FIG. 3, may be included in the theranostics GUI 1200. Each row of the flags column 1210 may include one or more flags and/or icons corresponding to patient details, for example lab results, imaging results, visit notes, and the like. For example, a lab icon 1222 may be included in the flags column 1210. In some examples, each flag and/or icon displayed within the flags column 1210 may indicate whether there is an abnormality detected within corresponding lab results, similar to as described with respect to FIG. 3. For example, the lab icon 1222 may be the second type of lab result flag that indicates that one or more abnormalities are detected within the corresponding lab results.

The flags and/or icons included in the flags column 1210 may be selectable elements that, when selected via one or more types of user inputs, modify the theranostics GUI 1200 to display pop-up GUIs (e.g., additional display panels) with additional information about the clement that was selected. As an example, the lab icon 1222 may be selectable via a mouse click or hover, to display additional details of lab results, including details of the detected abnormalities, as described with respect to FIG. 9.

Theranostics GUI 1200 may further include an alerts column 1212, wherein each row of the alerts column 1212 corresponds to a patient included in the theranostics GUI 1200. Each row of the alerts column 1212 may include one or more alert elements, such as first alert element 1224 in the first row. Alert elements in the alerts column 1212 may correspond to anticipated issues or risks for the designated theranostics protocol, reasons for candidacy for the designated theranostics protocol, or a track alert (e.g., a manually assigned alert) indicating to care providers that the corresponding patient is to be tracked for consideration for the designated theranostics protocol. For example, the first alert element 1224 indicates that the corresponding patient is a candidate for the designated theranostics protocol because of a lab result (e.g., a PMSA score for prostate cancer). Upon user selection of the first alert element 1224, additional information about the alert, such as additional information of the lab result (e.g., the PMSA score), a potential date for starting the designated theranostics protocol, or other information may be shown, or a task manager GUI may be displayed that allows the user to manage the alert.

The theranostics GUI 1200 may further comprise a completed tasks column 1214. The completed tasks column 1214 may include elements for tasks that have been completed for the corresponding patient. For example, completed task element 1226 may be displayed for the patient in the first row. The completed task element 1226, as an example, may indicate that the patient has completed imaging demanded to be a candidate for the designated theranostics protocol. If the imaging had yet to be completed, a corresponding element may, in some examples, be displayed as an alert element indicating that the patient has yet to complete imaging. Once completed, the completed task element corresponding to the imaging may be displayed in the completed tasks column 1214.

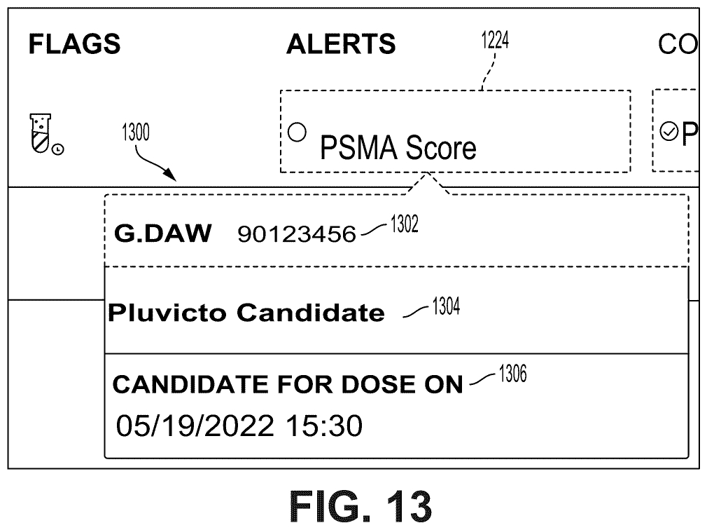

FIG. 13 shows an example eighth pop-up GUI 1300. The eighth pop-up GUI 1300 may be displayed in response to user selection, via a hover input for example, of the first alert clement 1224. The eighth pop-up GUI 1300 may comprise a patient information panel 1302 that includes a patient name and medical record number, in some examples, to identify the corresponding patient. The eighth pop-up GUI 1300 may include a patient status 1304 that indicates a status of the patient with respect to the designated theranostics protocol, such as “candidate” or “missing imaging” or “scheduled for protocol”. The eighth pop-up GUI 1300 may additionally comprise a potential dose date/time 1306 that indicates a potential date/time for the patient to start the designated protocol. In some examples, the potential dose date/time may not be included if the patient has yet to complete one or more steps, such as pre-therapy labs, imaging, or oncology visit, and may instead indicate which steps are to be completed prior to therapy.

The theranostics GUI 1200 and eighth pop-up GUI 1300 may allow users to easily visualize which patients are candidates for a designated theranostics protocol and to view a potential date for dose administration of a first therapy cycle. In this way, if any patients currently on the designated theranostics protocol have a delay, cancel an appointment, or are deemed unfit to continue with therapy, care providers may quickly and efficiently visualize which other patients are candidates and are ready to start therapy (e.g., have completed pre-therapy steps without step issues like lab abnormalities).

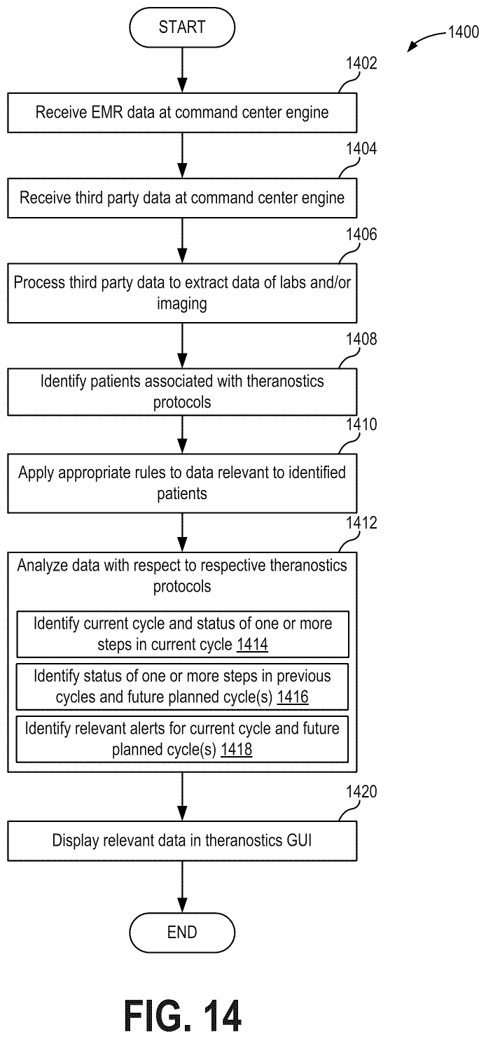

Turning now to FIG. 14, a flowchart illustrating a method 1400 for receiving and analyzing data to display in a theranostics GUI is shown. Method 1400 may be executed according to instructions stored in memory of a theranostics system of a computing device and executed by one or more processors of the computing device, such as computing device 102 of FIG. 1. As explained above, the computing device 102 may include a command center engine 140 that receives signals from one or more EMR databases (e.g., EMR database 280), analyzes the signals, and triggers alerts via theranostics engine 142 and/or theranostics engine 202, which are then displayed via an appropriate GUI, such as theranostics GUI 300 or theranostics GUI 1200.

At 1402, method 1400 includes receiving EMR data at the command center engine. The EMR data may include information bout one or more patients associated with a medical facility. The one or more patients may be assigned to or otherwise associated with one of a plurality of theranostics protocols or may not be assigned to or otherwise associated with one of the plurality of theranostics protocols. The EMR data may include information regarding patient vital signs, lab values, algorithmic scores, imaging data, outpatient visits, procedures including administrations of therapy, historical data (e.g., prior hospitalizations, prior diagnoses, prior orders, etc.), diagnoses, current medications, past medications, and scheduling data including scheduled dates and times of imaging exams, lab draws, therapy administrations, and outpatient visit appointments.

At 1404, method 1400 includes receiving third party data at the command center engine. The third party data may be received in a format that is not readable by the command center engine. For example, the third party data may comprise lab results scanned as a PDF. In such examples, the third party data may be scanned to memory for processing. The third party data may comprise lab information and/or imaging information.

At 1406, method 1400 includes processing the third party data to extract data of labs and/or imaging. As noted, the third party data may be in a format not readable by the command center engine, such as a scanned PDF. An OCR algorithm may be applied to extract individual characters from the third party data. An NLP algorithm may then be applied to the extracted characters to generate usable data of lab results and/or imaging results. Further, data of dates and times of collection, result, and/or exam may be extracted by the NLP algorithm. The third party data may be combined with the EMR data at the command center engine such that both the EMR data and the third party data are considered by the theranostics engine.

At 1408, method 1400 includes identifying patients on a theranostics protocol. The theranostics engine may be configured to recognize a plurality of theranostics protocols, such as protocols for prostate cancer, breast cancer, thyroid cancer, bone cancer, neuroendocrine cancer, and others. The protocols may include prescribed therapy courses, including prescribed or ordered medications, such as radiopharmaceuticals. Diagnoses, pathology findings, and/or current orders/prescriptions may indicate to the theranostics system that a patient is on a specified theranostics protocol or may be a candidate for a theranostics protocol. As a non-limiting example, a patient with a diagnosis of prostate cancer, a positive PMSA test, and current order for Pluvicto (a radiopharmaceutical) may be identified as a patient on a corresponding theranostics protocol. In contrast, a patient with a diagnosis of prostate cancer and a positive PMSA test but without a current order for Pluvicto may be identified as a candidate for the Pluvicto theranostics protocol.

At 1410, method 1400 includes applying appropriate rules to the received EMR data and processed third party data relevant to identified patients. As explained above with respect to FIG. 2, each respective theranostics protocols may have defined steps and rules to them. For example, a theranostics protocol for prostate cancer using Pluvicto may demand six cycles of treatment scheduled exactly 6 weeks apart or as close to exactly 6 weeks apart as possible. Applying the rules to analyze the received EMR data may include determining completed steps, scheduled steps, unscheduled steps, steps with issues including delays, etc. Applying the rules may also include defining various thresholds for dates, timelines, and lab values, in some examples.

At 1412, method 1400 includes analyzing the received EMR data and processed third party data with respect to respective theranostics protocols. Steps, cycles, and timeline demands of each respective theranostics protocol may be defined according to the rules, as noted at 1410. The received data for each patient on a theranostics protocol may comprise current orders (e.g., labs, imaging, medications, procedures, therapies), completed labs, completed imaging, scheduled imaging, scheduled office visits, and the like. The received data for each patient may be analyzed to determine which steps of a corresponding theranostics protocol are on target or at risk. For example, the received data may be analyzed to identify a current cycle and a status of one or more steps of the current cycle, as noted at 1414, and identify status of one or more steps in previous cycle(s) and future planned cycle(s), as noted at 1416. Further, relevant alerts for the current cycle and future planned cycle(s) may be identified, as noted at 1418. Identifying relevant alerts may include analyzing the received data to detect abnormal lab results, anticipated dose delays, unscheduled visits or therapy sessions, and uncollected labs, as non-limiting examples. Analyzing the data may also define which data for identified patients are relevant to the patient's respective theranostics protocol. For example, labs performed prior to diagnosis of prostate cancer may not be relevant, while labs performed as pre-therapy labs following diagnosis of prostate cancer may be relevant.

At 1420, method 1400 includes displaying relevant data in a theranostics GUI. As is presented in the above figures, the theranostics GUI may include data of one or more patients, including patient information, therapy dates/times, ordered labs, dates/times of scheduled outpatient oncology visits, imaging exams, and therapy procedures. Any identified relevant alerts may also be displayed within the theranostics GUI. The theranostics GUI may comprise selectable elements, as previously described, some of which when selected launch display of additional information, including lab data, imaging data, and the like.

The theranostics GUI may be updated in real-time and/or via manual request (e.g., a user input to an update element). In this way, data from the EMR databases and third party data may be iteratively sampled, processed, and analyzed for display within the theranostics GUI. For example, when data is updated automatically, data may be pulled from the various sources at a predefined frequency (e.g., every 1 minute, every 5 minutes) or in response to new data being available (e.g., via a push from a source). Therefore, users of the theranostics GUI may view and interact with the most up to date data of respective patients in order to properly assess status and risk for current and/or future theranostics protocol cycles.

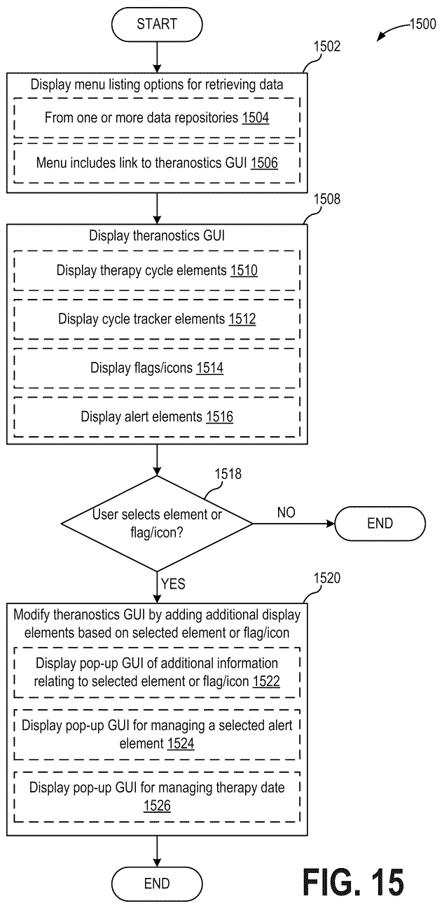

Referring now to FIG. 15, a flowchart illustrating a method 1500 for viewing and modifying a theranostics GUI is shown. Method 1500 may be implemented by the theranostics system in communication with one or more additional data repositories (e.g., computer systems and databases), such as one or more EMR systems. In some examples, aspects of method 1500 may be carried out by a theranostics system, such as theranostics system 200 of FIG. 2, according to instructions stored in memory (e.g., non-transitory memory 106) of the theranostics system, which may be executed by one or more processors (e.g., processor 104) of the theranostics system.

At 1502, method 1500 includes displaying a menu listing options for retrieving data. The data may include data of one or more patients and may be obtained from a plurality of databases and systems of a hospital as well as from third parties, in some examples. Ass indicated at 1504, the options listed in the menu may include an option to retrieve data from one or more data repositories, such as an EMR system or a third party. Further, the options listed in the menu may include an option to display a theranostics GUI, and thus the menu may include a link to the theranostics GUI, as indicated at 1506. In some examples, the theranostics GUI may be accessed from the one or more data repositories. For example, a user may launch an EMR interface whereby one or more EMRs of a patient may be viewed. The EMR interface may include a link to the theranostics GUI that, when selected, launches the theranostics GUI. In some examples, the menu may include a command center or hospital systems menu whereby the theranostics GUI and the EMR interface may be accessed via separate menu options.

At 1508, method 1500 includes displaying the theranostics GUI. As explained previously, the theranostics GUI may be displayed in response to selection of a link, which may be displayed via the EMR interface, a command center interface, a hospital systems menu, or other suitable interface. The theranostics GUI may display elements for one or more patients. For example, the one or more patients may be patients currently associated with a designated theranostics protocol, all patients on any theranostics protocol, or patients associated with a theranostics protocol who have one or more cycles at risk. What subset of patients is displayed within the theranostics GUI may be determined by a selected or a default profile.

The theranostics GUI may display respective therapy cycle elements, as noted at 1510, for each displayed patient. The therapy cycle elements, as described with respect to FIG. 3, may indicate a date/time and status of a scheduled therapy procedure. Each element may comprise an information panel detailing the cycle number and the date and time and each element may be displayed with a color representative of the status (e.g., green if on target, red if at risk, etc.). For example, planned sixth therapy cycle element 332 as shown in FIG. 3 comprises the date panel 334 that indicates a date and time for which the planned sixth therapy cycle is scheduled for the corresponding patient.

The theranostics GUI may further display cycle tracker elements for each displayed patient, as noted at 1512. Each theranostics protocol may have a defined number of cycles and the theranostics GUI may display the defined number of cycle tracker elements for respective patients. For example, for patients on a Pluvicto theranostics protocol which demands six cycles of therapy, six cycle tracker elements may be displayed, such as cycle two element 372 of FIG. 3. The theranostics GUI may further display one or more flags or icons, such as lab flab 338 and second lab flag 374, as noted at 1514. The flags or icons may be selectable elements that indicate a variety of information. For example, lab flags may indicate whether any abnormal lab results have been detected based on a color of the flag, snooze icons may indicate whether a current element (e.g., an alert element) has been temporarily snooze, an escalate icon may indicate whether a current element has been escalated to another user or group of users, and so on.

The theranostics GUI may additionally display one or more alert elements for each displayed patient, as noted at 1516, when one or more issues have been detected within the patient data. For example, a lab abnormality may trigger display of an alert element, such as second alert element 376, for the lab abnormality and an anticipated delay in delivery a therapy dose may trigger display of an alert element, such as first alert element 352, for the delay. As described with respect to the above figures, each of the therapy cycle elements, cycle tracker elements, flags/icons, and alert elements may be selectable elements that, when selected via one or more types of user inputs, launch a pop-up GUI.

At 1518, method 1500 determines if a user has selected an element, flag, or icon. The user (which may be a care provider) may enter a user input to an element, flag, or icon, such as a mouse or touch pad hover, a mouse click or touch pad input (e.g., tap or click), in order to view additional information associated with the element, flag, or icon or to manage a task, such as an alert or schedule, associated with the element, flag, or icon. In some examples, different types of user inputs to the same element, flag, or icon may trigger different GUI modifications.

At 1520, method 1500 includes modifying the theranostics GUI by adding additional information based on the selected element, flag, or icon. The additional information may include, as indicated at 1522, a pop-up GUI of additional information relating to the selected element, flag, or icon, such as second pop-up GUI 500, third pop-up GUI 600, fourth pop-up GUI 650, fifth pop-up GUI 700, seventh pop-up GUI 900, and/or eighth pop-up GUI 1300. As an example, a pop-up GUI that is launched in response to user selection of a cycle tracker element may display a plurality of step elements with information panels corresponding to respective steps.

The additional information may alternatively include, as indicated at 1524, a pop-up GUI for managing a selected alert element. In some examples, a first type of user input, such as a hover, selecting an alert clement may launch a pop-up GUI displaying additional information of the alert element, such as fifth pop-up GUI 700, while a second type of user input, such as a mouse click, selecting the same alert element may launch a pop-up GUI for managing the alert element, such as sixth pop-up GUI 800. Pop-up GUIs for managing alert elements may display each of one or more alerts for a corresponding patient and may allow the user to take various actions for each alert, such as acknowledging the alert, snoozing the alert, and so on as previously described.

The additional information may alternatively include, as indicated at 1526, a pop-up GUI for managing therapy date. In some examples, user input to a therapy cycle element may launch display of a pop-up GUI, such as first pop-up GUI 400, that includes various selectable options for scheduling the corresponding therapy cycle. The patient specific information displayed in any of the optional pop-up GUIs described herein may be obtained from the patient's EMR and/or third parties stored within one or more data repositories (e.g., one or more EMR systems).

When the theranostics GUI is displayed and/or when the additional display elements (e.g., the limited set of visual elements and/or the various pop-up GUIs described herein) are displayed, the one or more EMRs may exist in an un-launched state (e.g., not displayed and not currently accessed by the care provider device.) In this way, the data from the EMRs may be used to generate the theranostics GUI and the pathway tracker and alert elements displayed therein and the EMR data may be used to populate additional display panels (e.g., pop-up GUIs) without the user having to access the EMRs themselves, for example in a separate window. In doing so, the theranostics GUI as herein disclosed may present a limited set of information, including a pathway tracker with steps of each cycle, indication of status of each step, and alerts for data that may affect status of a step to the users/care providers in order to increase efficiency of the users' interaction with the available data as users are not forced to sift through multiple separate EMRs, scanned data from third parties, and multiple schedules in order to determine whether a theranostics protocol is on track.

The systems, methods, and graphical user interfaces provided herein may improve the accuracy and efficiency of monitoring of patients who are on or who may be candidates for theranostics protocols. Theranostics protocols demand many steps set up in a highly coordinated manner. By collecting data from multiple data repositories and including third party data that is processed into a usable format, schedules, results, and potential risks for a theranostics protocol of a patient may be more easily viewed and digested by care providers. The time it would take to individually collect data from multiple EMRs, aggregate and analyze the collected data, and visualize the data using standard methods could render the data useless by the time the data was seen as theranostics protocols often have specific timeframes. By establishing a system that automatically receives data from all EMRs in real time or near real time, aggregates that information regardless of data format, and continually updates a theranostics GUI as data is received, the approach of the disclosure allows care providers to make informed decisions about theranostics protocols, including decisions in scenarios when delays are anticipated or imaging/labs/office visits are not scheduled or therapy procedures are scheduled outside the demanded timeline of the theranostics protocol.

In contrast, in prior systems when a care provider attempted to determine whether a patient on a theranostics protocol has completed all steps prior to a therapy procedure for the protocol, the care provider may need to scour multiple data repositories, call various offices, and track down scanned reports in order to determine pre-therapy lab results and scheduled office visits and to determine whether the scheduled therapy procedure is in a correct timeframe. This approach for prior systems is time consuming and prone to errors, including human errors or delays. Further, even in a most time efficient effort of prior systems, multiple data repositories have to be accessed and multiple displays have to be rendered by the computing device. The disclosure provides a specific way of improving the capability of the healthcare system by providing one or more theranostics GUI that display dynamically updated data of patients associated with theranostics protocols. The disclosure further provides a specific improvement to the way computers operate by aggregating EMR data for multiple patients in one location and updating the patient data respective to a corresponding theranostics protocol, which may obviate the need for users to have to navigate through multiple different EMRs/data files, manually update information as data and statuses change, and so forth, thereby increasing efficiency of the operation of the computer for the user.

The theranostics GUIs herein described provide a specific manner of displaying a limited set of information to a user, including the pathway tracker and displayed alerts, rather than using conventional user interface methods to display a generic index on a computer, requiring the user to step through many layers of menu options to access the desired data, or burying the desired data within all hospital data. Thus, the user experience with the computer may be improved and made more efficient.

Furthermore, by displaying a limited set of information via the theranostics GUIs as described herein, operation of the computing device(s) that collect and render the data for display may be improved by reducing the processing demands of the computing device(s), thereby increasing the efficiency of the computing device(s). For example, only certain lab results leading to an “at risk” indication for a step of a cycle of a theranostics protocol may be displayed, which results in a limited amount of the data that is received being processed, which may improve the efficiency of the computing device(s). Further, in some examples, the data is processed in real time and updates to the theranostics GUIs are made continuously as data is received, and therefore undue processing lags that occur if the updates were made at predefined time points may be reduced, which may improve the efficiency of the computing device(s). Additionally, as described herein, if network lags or outages result in an incomplete pathway tracker element, users may be notified that the pathway tracker is not available rather than being presented with an incomplete and hence potentially inaccurate pathway tracker.

The theranostics system, included as a part of a medical information system as described herein, may provide for high speed data processing that allows for the real time or near real time updates to the theranostics GUIs. For example, the theranostics system may be configured to update the theranostics GUIs every 30 seconds, rather than every 15 minutes or longer as in some prior systems.

Thus, via the disclosed theranostics system, status of cycles and cycle steps for patients on theranostics protocols may be displayed in a manner that is easy to visually parse and act on in a reduced amount of time. The patient information may be displayed via small graphical elements with minimal text, which may allow a large number of elements to be included on the same screen. A user may then select a graphical element of interest to view more information about a corresponding event, record, or report and/or to manage a schedule or task associated with the graphical clement. The patient information may be stored in different data repositories that would otherwise be accessed via individual interfaces, and by using the theranostics system to aggregate the patient information, an amount of time necessary to review relevant patient information for diagnosis and treatment decisions may be reduced. The theranostics system disclosed herein may also aggregate patient data to a single place, into a single application, which may help to reduce wasted time spent searching for known but scattered data, and unknown and missing data. This may reduce cognitive overloads and aid clinical thinking; because the patient data is reconstructed into a clinically helpful structure. The technical effect of automatically generating alerts and presenting cycle step elements with various colors that indicate a status thereof for patients on theranostics protocols by applying a set of rules to selected patient data is that errors or delays due to sorting through large amount of patient data may be reduced, and an amount of time spent by a clinician making decisions may be reduced.

The disclosure also provides support for a computing device comprising a display screen, the computing device being configured to display on the display screen a menu listing one or more electronic medical records (EMRs) of one or more patients, and additionally being configured to display on the display screen a theranostics graphical user interface (GUI) accessible from the menu, wherein the theranostics GUI displays, for each patient, a pathway tracker indicating status of a corresponding theranostics protocol, wherein the pathway tracker comprises, for each cycle of the corresponding theranostics protocol, a limited snapshot of statuses of steps thereof, wherein each limited snapshot is selectable to launch a pop-up GUI with additional information relating to each of the steps thereof, and wherein the theranostics GUI is displayed while the one or more EMRs are in an un-launched state. In a first example of the system, the pathway tracker includes an element that indicates a status of a current cycle of the corresponding theranostics protocol. In a second example of the system, optionally including the first example, the status of the current cycle is one of on track and at risk based on data obtained from the one or more EMRs. In a third example of the system, optionally including one or both of the first and second examples, the pop-up GUI includes a plurality of step elements, wherein each of the plurality of step elements includes an information panel detailing one or more of a scheduled date, a completion date, and an issue of a corresponding step based on one or more patient parameters received from the one or more EMRs. In a fourth example of the system, optionally including one or more or each of the first through third examples, the determined status of the corresponding step is one of on track, at risk, completed, and irrelevant and wherein the determined status of the corresponding step is determined based on one or more patient parameters received from the one or more EMRs. In a fifth example of the system, optionally including one or more or each of the first through fourth examples, the status of the current cycle is at risk when one or more of the plurality of step elements indicate the determined status of the corresponding step is at risk. In a sixth example of the system, optionally including one or more or each of the first through fifth examples, the theranostics GUI further displays one or more selectable elements for each patient, each of the one or more selectable elements being selectable to launch a second pop-up GUI. In a seventh example of the system, optionally including one or more or each of the first through sixth examples, the one or more selectable elements include one or more of alert elements, therapy cycle elements indicate scheduled dates of therapy cycles, flags, and icons. In a eighth example of the system, optionally including one or more or each of the first through seventh examples, the second pop-up GUI, when launched in response to a first type of user input to a selectable element, displays additional information relating to the selectable element. In a ninth example of the system, optionally including one or more or each of the first through eighth examples, the second pop-up GUI, when launched in response to a second type of user input to a selectable clement, displays one or more management elements that when selected manage the selectable element.

The disclosure also provides support for a method for a theranostics system, comprising: displaying a menu listing one or more options for retrieving data of a plurality of patients from a plurality of data repositories of a hospital system, the plurality of data repositories including one or more electronic medical record (EMR) systems, displaying a theranostics graphical user interface (GUI) that displays, for one or more of the plurality of patients, a pathway tracker indicating a first status of a current cycle of a theranostics protocol determined from the retrieved data from the one or more EMR systems, wherein the pathway tracker includes a snapshot clement for each cycle of the theranostics protocol, and in response to a user selection of the snapshot clement for a selected cycle, adding additional display elements to the theranostics GUI, the additional display elements identifying a second status of each step of the selected cycle determined from the retrieved data from the one or more EMR systems, wherein the theranostics GUI is displayed while the one or more EMR systems are in an un-launched state. In a first example of the method, the first status of the current cycle of the theranostics protocol is determined based on a set of rules applied to the retrieved data, the set of rules identifying demanded steps of the theranostics protocol and the second status of each step of the current cycle. In a second example of the method, optionally including the first example, the theranostics GUI includes, for a first patient, an alert element indicating a detected issue with a step of a cycle of the theranostics protocol. In a third example of the method, optionally including one or both of the first and second examples, the alert element is selectable to add a second additional display element to the theranostics GUI, the second additional display element including information specific to the alert obtained from the retrieved data from the one or more EMR systems.

The disclosure also provides support for a theranostics system, comprising: one or more processors, and memory storing instructions executable by the one or more processors to: receive, at the theranostics system, a feed from an electronic medical record (EMR) database, where one or more patient parameters for each of a plurality of patients are sent over the feed, identify one or more patients of the plurality of patients associated with a theranostics protocol based on the received feed, output, for display on a display device, a theranostics graphical user interface (GUI) that includes, for the one or more patients of the plurality of patients, a pathway tracker comprising a plurality of cycle elements, wherein each of the plurality of cycle elements is selectable and displays a snapshot of steps of a given cycle, wherein each snapshot of steps is generated by applying a set of rules specific to the theranostics protocol to a set of patient data obtained from the feed, and display, on the theranostics GUI, for a selected patient of the one or more patients, a risk element that indicates whether a current cycle of the theranostics protocol is at risk. In a first example of the system, each of the snapshot of steps is a selectable element that, when selected, launches a pop-up GUI displaying a plurality of step elements, wherein each of the plurality of step elements is displayed in a color corresponding to a status thereof. In a second example of the system, optionally including the first example, a first step element of the plurality of step elements is displayed with a first color indicating that a corresponding first step is at risk. In a third example of the system, optionally including one or both of the first and second examples, a second step element of the plurality of step elements is displayed with a second color indicating that a corresponding second step is on target. In a fourth example of the system, optionally including one or more or each of the first through third examples, the instructions are executable to display, on the theranostics GUI, a lab flag indicating detection of a lab abnormality. In a fifth example of the system, optionally including one or more or each of the first through fourth examples, data in the feed comprises data obtained from the EMR database and data obtained from one or more third parties, wherein the data obtained from the one or more third parties is processed via optical character recognition and natural language processing to reformat the data.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A computing device comprising a display screen, the computing device being configured to display on the display screen a menu listing one or more electronic medical records (EMRs) of one or more patients, and additionally being configured to display on the display screen a theranostics graphical user interface (GUI) accessible from the menu, wherein the theranostics GUI displays, for each patient, a pathway tracker indicating status of a corresponding theranostics protocol, wherein the pathway tracker comprises, for each cycle of the corresponding theranostics protocol, a limited snapshot of statuses of steps thereof, wherein each limited snapshot is selectable to launch a pop-up GUI with additional information relating to each of the steps thereof, and wherein the theranostics GUI is displayed while the one or more EMRs are in an un-launched state.

2. The computing device of claim 1, wherein the pathway tracker includes an element that indicates a status of a current cycle of the corresponding theranostics protocol.

3. The computing device of claim 2, wherein the status of the current cycle is one of on track and at risk based on data obtained from the one or more EMRs.

4. The computing device of claim 3, wherein the pop-up GUI includes a plurality of step elements, wherein each of the plurality of step elements includes an information panel detailing one or more of a scheduled date, a completion date, and an issue of a corresponding step based on one or more patient parameters received from the one or more EMRs.

5. The computing device of claim 4, wherein the determined status of the corresponding step is one of on track, at risk, completed, and irrelevant and wherein the determined status of the corresponding step is determined based on one or more patient parameters received from the one or more EMRs.

6. The computing device of claim 4, wherein the status of the current cycle is at risk when one or more of the plurality of step elements indicate the determined status of the corresponding step is at risk.

7. The computing device of claim 1, wherein the theranostics GUI further displays one or more selectable elements for each patient, each of the one or more selectable elements being selectable to launch a second pop-up GUI.

8. The computing device of claim 7, wherein the one or more selectable elements include one or more of alert elements, therapy cycle elements indicate scheduled dates of therapy cycles, flags, and icons.

9. The computing device of claim 7, wherein the second pop-up GUI, when launched in response to a first type of user input to a selectable element, displays additional information relating to the selectable element.

10. The computing device of claim 7, wherein the second pop-up GUI, when launched in response to a second type of user input to a selectable element, displays one or more management elements that when selected manage the selectable element.

11. A method for a theranostics system, comprising:
displaying a menu listing one or more options for retrieving data of a plurality of patients from a plurality of data repositories of a hospital system, the plurality of data repositories including one or more electronic medical record (EMR) systems;
displaying a theranostics graphical user interface (GUI) that displays, for one or more of the plurality of patients, a pathway tracker indicating a first status of a current cycle of a theranostics protocol determined from the retrieved data from the one or more EMR systems, wherein the pathway tracker includes a snapshot element for each cycle of the theranostics protocol; and
in response to a user selection of the snapshot element for a selected cycle, adding additional display elements to the theranostics GUI, the additional display elements identifying a second status of each step of the selected cycle determined from the retrieved data from the one or more EMR systems, wherein the theranostics GUI is displayed while the one or more EMR systems are in an un-launched state.

12. The method of claim 11, wherein the first status of the current cycle of the theranostics protocol is determined based on a set of rules applied to the retrieved data, the set of rules identifying demanded steps of the theranostics protocol and the second status of each step of the current cycle.

13. The method of claim 11, wherein the theranostics GUI includes, for a first patient, an alert element indicating a detected issue with a step of a cycle of the theranostics protocol.

14. The method of claim 13, wherein the alert element is selectable to add a second additional display element to the theranostics GUI, the second additional display element including information specific to the alert obtained from the retrieved data from the one or more EMR systems.

15. A theranostics system, comprising:
one or more processors; and
memory storing instructions executable by the one or more processors to:
receive, at the theranostics system, a feed from an electronic medical record (EMR) database, where one or more patient parameters for each of a plurality of patients are sent over the feed;
identify one or more patients of the plurality of patients associated with a theranostics protocol based on the received feed;
output, for display on a display device, a theranostics graphical user interface (GUI) that includes, for the one or more patients of the plurality of patients, a pathway tracker comprising a plurality of cycle elements, wherein each of the plurality of cycle elements is selectable and displays a snapshot of steps of a given cycle, wherein each snapshot of steps is generated by applying a set of rules specific to the theranostics protocol to a set of patient data obtained from the feed; and
display, on the theranostics GUI, for a selected patient of the one or more patients, a risk element that indicates whether a current cycle of the theranostics protocol is at risk.

16. The theranostics system of claim 15, wherein each of the snapshot of steps is a selectable element that, when selected, launches a pop-up GUI displaying a plurality of step elements, wherein each of the plurality of step elements is displayed in a color corresponding to a status thereof.

17. The theranostics system of claim 16, wherein a first step element of the plurality of step elements is displayed with a first color indicating that a corresponding first step is at risk.

18. The theranostics system of claim 16, wherein a second step element of the plurality of step elements is displayed with a second color indicating that a corresponding second step is on target.

19. The theranostics system of claim 15, wherein the instructions are executable to display, on the theranostics GUI, a lab flag indicating detection of a lab abnormality.

20. The theranostics system of claim 15, wherein data in the feed comprises data obtained from the EMR database and data obtained from one or more third parties, wherein the data obtained from the one or more third parties is processed via optical character recognition and natural language processing to reformat the data.
